(19) United States
(12) Patent Application Publication
(10) Pub. No.: US 2008/0098780 A1
(43) Pub. Date: May 1, 2008

(54) SYSTEMS AND METHODS FOR TREATING SOLID WASTE

(75) Inventor: Don Shubin, Tustin, CA (US)

Correspondence Address: KNOBBE MARTENS OLSON & BEAR LLP, 2040 MAIN STREET, FOURTEENTH FLOOR, IRVINE, CA, 92614, US

(73) Assignee: Federal Disposal Services, Inc., Tustin, CA (US)

(21) Appl. No.: 11/876,672

(22) Filed: Oct. 22, 2007

Related U.S. Application Data
(60) Provisional application No. 60/853,361, filed on Oct. 20, 2006.
(60) Provisional application No. 60/888,498, filed on Feb. 6, 2007.
(60) Provisional application No. 60/916,793, filed on May 8, 2007.

(51) Int. Cl. C05F 11/08; C12M 1/02

(52) U.S. Cl. 71/9; 435/290.2

(57) ABSTRACT

A system and method for treating solid waste includes a trommel screen and a composting unit. The trommel screen includes an inlet hopper and a rotating screening drum, which can be equipped with internal channeling members and bag rippers with blades. A composting and/or dewatering apparatus has a vessel portion, a cover portion and an internal auger. The apparatus includes a plurality of openings along its vessel portion to permit a volume of liquid from within the composting apparatus to be eliminated. The composting/dewatering unit can also include a sleeve member along the outside of the vessel portion. In one embodiment, waste items passing through the screening drum of the trommel screen are conveyed into the composting unit. In another embodiment, the discharged liquid is collected in one or more liquid collection structures and is conveyed to a clarifier tank.

### CROSS-REFERENCE TO RELATED APPLICATIONS

This application claims the priority benefit under 35 U.S.C. § 119(e) of U.S. Provisional Application No. 60/853,361, filed Oct. 20, 2006, U.S. Provisional Application No. 60/888,498, filed Feb. 6, 2007 and U.S. Provisional Application No. 60/916,793, filed May 8, 2007, the entirety of all are hereby incorporated by reference herein.

### BACKGROUND

1. Field of the Invention

This application relates to devices, systems and methods of handling waste, and more specifically, to devices, systems and methods for screening, sorting, mixing and/or composting solid waste, including sludge, municipal waste, fertilizer, dairy waste and the like.

2. Description of the Related Art

Trommel screens are typically used in material recovery and other facilities to separate items by size and/or material type. Material fed into trommel screens can either pass through the screen portion or move to the downstream end of the rotating screening drum. Thus, trommel screens can be used to sort items according to size.

Composting is the process by which microbes and other microorganisms decompose organic and other biodegradable materials. Although composting can occur naturally, controlled composting provides an environment in which decomposers can thrive, thereby speeding up the decomposition process. Typically, a correct mix of carbon, nitrogen, oxygen and water is necessary to enhance a controlled composting process. For example, carbon sources provide cellulose which composting bacteria convert into sugars and heat, whereas, nitrogen sources provide protein which permits composting bacteria to thrive. Thus, the carbon to nitrogen ratio and the presence of certain organic or inorganic substances and other characteristics of the materials fed into a composting unit are often regulated in an attempt to make the composting process more efficient. In addition, other parameters, such as, for example, operating temperature, air flow, moisture content, mixing rate and the like may also be used to further control the decomposition rate and other aspects of the composting process.

### SUMMARY

According to some embodiments, an apparatus for dewatering and composting a volume of waste includes a vessel portion comprising an upper end, an exterior surface, an interior surface and a plurality of openings, the openings extending from the interior surface to the exterior surface. The apparatus further comprises a cover portion attached to the upper end of the vessel portion, the cover portion and vessel portion defining an interior cavity, and one or more sleeve portions positioned at least partially along the exterior surface of the vessel portion. The sleeve portion includes an inlet, the sleeve portion and the exterior surface defining a space. The apparatus further comprises at least one mixing member positioned within the interior cavity. In some embodiments, at least some of the openings in the vessel portion are in fluid communication with the space. Further, the openings are configured to permit fluids to discharge therethrough, both from the interior cavity to the space and from the space to the interior cavity. According to other embodiments, a dewatering system for use in a composting apparatus includes a vessel portion having side walls and a bottom surface, at least one of the side walls or the bottom surface of the vessel portion comprising a plurality of openings configured to permit a liquid to discharge therethrough and at least one liquid collection member configured to collect liquid discharged from the openings. The operation of a mixing member within an interior of the vessel portion facilitates the flow of liquid through the openings.

In some embodiments, a system for treating waste materials includes a trommel screen and a composting apparatus. The trommel screen comprises an inlet hopper and a screening drum comprising a plurality of openings. The screening drum is configured to receive a volume of waste materials from the inlet hopper and to separate the volume of waste materials into at least a first waste type and a second waste type, the first waste type passing through the plurality of openings. Further, the composting apparatus comprises a vessel portion having a plurality of orifices configured to permit a liquid to discharge therethrough, a cover portion attached to an upper section of the vessel portion, the cover portion and the vessel portion defining an interior space, and at least one mixing member positioned within the interior space.

In one embodiment, a system for treating waste materials includes a trommel screen and a composting unit. The trommel screen can include an inlet hopper and a screening drum. The composting apparatus can include a vessel portion having a plurality of openings configured to permit a liquid to discharge therethrough. In addition, the composting apparatus can include a cover portion attached to an upper section of the vessel portion. The cover portion and vessel portion define an interior space of the composting unit. Further, the composting unit comprises one or more mixing members positioned within its interior space.

In some embodiments, a method of dewatering a volume of waste materials placed situated within a composting apparatus comprises providing a composting apparatus, the composting apparatus comprising a vessel portion and a cover member situated generally above the vessel portion, the vessel portion and the cover member defining an interior cavity, wherein the vessel portion comprises an interior surface and a plurality of openings being in fluid communication with the interior cavity. The method further includes operating at least one mixing member, the mixing member having an axis and being positioned at least partially within the interior cavity, the mixing member comprising a lower base portion, the lower base portion comprising at least one baffle. In addition, the method comprises accessing a volume of waste materials by opening a closure member situated along the vessel portion. In some embodiments, operating the mixing member causes a volume of waste materials to be compressed against a portion of the interior surface of the vessel portion.

In another embodiment, the trommel screen comprises at least one conveyor, which is configured to transfer a volume of solids away from the screening drum. In other embodiments, the system additionally comprises a clarifier tank, which is configured to receive a volume of liquid removed from the composting unit. In yet another embodiment, the clarifier tank comprises at least one membrane, which is configured to separate a volume of liquid from a volume of solids.

In one embodiment, a trommel screen for screening waste materials includes an inlet hopper and a screening drum. The screening drum can be configured to receive waste materials from the inlet hopper and deliver them to a downstream end of the drum screen or dispose of them through a screen surface of the screening drum. In another embodiment, the inlet hopper of the trommel screen comprises a conveyor.

In one embodiment, the screening drum of the trommel screen includes one or more channeling members along its interior. In another embodiment, the channeling members can be configured to facilitate the movement of waste materials through the screening drum. In other embodiments, a channeling member can be a metal flat bar.

In one embodiment, the screening drum of the trommel screen includes at least one bag ripper. In other embodiments, the bag ripper comprises one or more blades. In yet other embodiments, an interior portion of the screening drum comprises a protective coating. In one embodiment, the protective coating includes polyurethane, epoxy, plastic or the like.

In one embodiment, a composting and/or dewatering apparatus designed to compost a volume of waste comprises a vessel portion having a plurality of openings configured to permit a liquid to discharge therethrough and a cover portion attached to an upper section of the vessel portion, the cover portion and vessel portion defining an interior space. The composting apparatus further includes one or more mixing members positioned within the interior space. In one embodiment, the composting apparatus further includes a liquid collection member positioned along an exterior area of the vessel portion and configured to receive a volume of liquid discharged from the openings. In some embodiments, the mixing member includes one or more augers. In one embodiment, the auger is substantially cone-shaped. In other embodiments, the mixing member includes a vertically-oriented auger located near the center of the interior space.

In one embodiment, the vessel portion includes substantially cylindrically-shaped side walls and a substantially horizontally-oriented bottom wall. In another embodiment, the side walls taper inwardly closer to the bottom wall. In other embodiments, the composting apparatus further comprises one or more hatches positioned along an area of the vessel portion. Such hatches are configured to permit access to an interior space of the composting apparatus. In yet another embodiment, the composting apparatus further includes a condensation collection member positioned near the interface of the vessel portion and the cover portion. The condensation collection member may be configured to collect condensation developing on a surface of the cover member.

In one embodiment, the composting apparatus further comprises a sediment hopper located near a bottom area of the interior space, the sediment hopper being configured to receive a volume of sand, grit, silt and/or other heavier solids.

In other embodiments, the cover portion comprises at least one cover opening. In some embodiments, the vessel portion and the cover portion are a unitary member. In another embodiment, the vessel portion and the cover portion are separate members, with the cover portion configured to be removed from the vessel portion. In yet another embodiment, the cover portion comprises a flexible material. In some embodiments, the cover portion is manufactured from plastic, rubber, tarp, metal and/or other materials.

In one embodiment, the composting apparatus further comprises a clarifier tank which is configured to receive a volume of liquid collected by one or more liquid collection members. In another embodiment, the clarifier tank includes one or more membranes and/or other filters, the membranes or other members being configured to separate a volume of liquid from a volume of solids. In other embodiments, the clarifier tank is configured to chemically, biologically and/or otherwise treat the liquid collected therein.

### BRIEF DESCRIPTION OF THE DRAWINGS

These and other features, aspects and advantages of certain embodiments of the present invention are described with reference to drawings, which are intended to illustrate, but not to limit, the disclosure herein. The drawings include thirty-eight (38) figures. It is to be understood that the attached drawings are for the purpose of illustrating concepts and may not be to scale.

FIG. 1 is a schematic flow chart of a solid waste handling, sorting and treatment system according to one embodiment that is arranged and configured in accordance with certain features, aspects and advantages of the present invention;

FIG. 2 is a side elevation view of a trommel screen according to one embodiment that is arranged and configured in accordance with certain features, aspects and advantages of the present invention;

FIG. 3 is a detailed side elevation view of the inlet hopper and screening drum of the trommel screen of FIG. 2;

FIG. 4 is a perspective view of a portion of the screening drum of FIG. 3;

FIG. 5A is a detailed view of the screening surface of a drum according to one embodiment that is arranged and configured in accordance with certain features, aspects and advantages of the present invention;

FIG. 5B is a cross-sectional view of the screening drum taken along line 5B-5B of FIG. 3;

FIG. 5C is a detailed view of a knife member of the screening drum according to one embodiment that is arranged and configured in accordance with certain features, aspects and advantages of the present invention;

FIG. 6 is a front elevation view of an in-vessel dewatering/composting apparatus according to one embodiment that is arranged and configured in accordance with certain features, aspects and advantages of the present invention;

FIG. 7 is a detailed cross-sectional view of the interface between the vessel body and the cover of the dewatering/composting apparatus of FIG. 6 taken along 7-7;

FIG. 8A is a frontal elevation view of a dewatering/composting apparatus with a portion of the vessel body cut away to reveal an internal auger according to one embodiment that is arranged and configured in accordance with certain features, aspects and advantages of the present invention;

FIG. 8B is a frontal elevation view of a dewatering/composting apparatus with a portion of the vessel body cut away to reveal an internal auger according to another embodiment that is arranged and configured in accordance with certain features, aspects and advantages of the present invention;

FIG. 9 is a detailed perspective view of a liquid collection structure according to one embodiment that is arranged and configured in accordance with certain features, aspects and advantages of the present invention;

FIG. 10 is a cross-sectional view of a clarifier tank according to one embodiment that is arranged and configured in accordance with certain features, aspects and advantages of the present invention;

FIG. 11 is a front elevation view of a dewatering/composting apparatus according to another embodiment that is arranged and configured in accordance with certain features, aspects and advantages of the present invention;

FIG. 12A is a bottom view of the dewatering/composing apparatus of FIG. 11;

FIG. 12B is a bottom view of a dewatering/composting apparatus according to another embodiment that is arranged and configured in accordance with certain features, aspects and advantages of the present invention;

FIG. 12C is a cross-sectional view of the dewatering/composting apparatus of FIG. 12B, taken along 12C-12C;

FIG. 13A is front elevation view of a dewatering/composting apparatus with a portion of the outer sleeve cut away to reveal the main vessel wall according to still another embodiment that is arranged and configured in accordance with certain features, aspects and advantages of the present invention;

FIG. 13B is a cross-sectional view of the dewatering/composting apparatus of FIG. 13A, taken along 13B-13B;

FIG. 14A is a top view of a dewatering/composting apparatus comprising two internal augers according to one embodiment that is arranged and configured in accordance with certain features, aspects and advantages of the present invention;

FIG. 14B is a top view of a dewatering/composting apparatus comprising a single internal auger according to one embodiment that is arranged and configured in accordance with certain features, aspects and advantages of the present invention;

FIG. 14C is a side elevation view of an internal portion of the dewatering/composting apparatus of FIG. 14A, taken along 14C-14C;

FIG. 15A is a front elevation view of a dewatering/composting apparatus with a portion of its cover portion cut away to reveal an odor control system according to one embodiment that is arranged and configured in accordance with certain features, aspects and advantages of the present invention;

FIG. 15B is a front elevation view of a dewatering/composting apparatus configured to be connected to an odor control system according to another embodiment that is arranged and configured in accordance with certain features, aspects and advantages of the present invention;

FIG. 15C is a cross-sectional view of an odor control system configured to treat the fluid discharged from a dewatering/composting apparatus according to one embodiment that is arranged and configured in accordance with certain features, aspects and advantages of the present invention;

FIG. 16A illustrates an elevation view of a dewatering/compositing apparatus with a portion of an outer sleeve cut away to reveal the main vessel wall according to one embodiment;

FIG. 16B illustrates a bottom view of the lower surface of the dewatering/composting apparatus of FIG. 16A;

FIG. 16C illustrates a cross-sectional view of the main vessel wall of the dewatering/composting apparatus of FIG. 16A;

FIG. 17A illustrates a cross-sectional view of a dewatering/composting apparatus according to one embodiment;

FIG. 17B illustrates a top view of the dewatering/composting apparatus of FIG. 17A;

FIG. 17C illustrates a side view of the wing portion of the auger of the dewatering/composting apparatus of FIG. 17A;

FIG. 17D illustrates an interior elevation view of the dewatering/composting apparatus of FIG. 17A;

FIG. 17E illustrates a cross-sectional view of the sidewall of the dewatering/composting apparatus of FIG. 17A;

FIG. 18 schematically illustrates a dewatering/composting apparatus comprising downstream treatment processes in accordance with one embodiment; and

FIGS. 19A-19D illustrate a schematic process flow diagram for treating solid and liquid waste and providing odor control according to one embodiment that is arranged and configured in accordance with certain features, aspects and advantages of the present inventions.

### DETAILED DESCRIPTION OF THE PREFERRED EMBODIMENTS

The discussion below and the figures referenced herein describe various embodiments of an apparatus and method for screening, separating, composting and/or dewatering and/or otherwise treating waste. A number of these embodiments are particularly well suited for implementation in a trommel screen, vertical enclosed vessel composting unit and/or the like. However, it will be appreciated that the features, aspects and advantages related to the different embodiments described herein may be incorporated into other types of screening units, composting units, dewatering units and other treatment apparatuses.

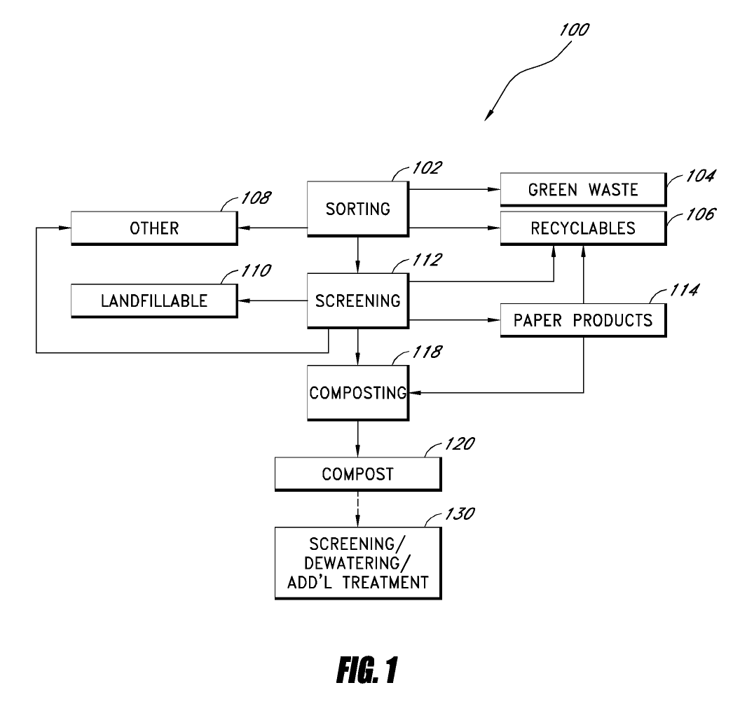

FIG. 1 schematically illustrates a solid waste treatment system 100 in accordance with one embodiment of the present inventions. As shown, the system 100 can comprise an initial sorting step 102 during which waste items may be separated into different bins. The sorting can be accomplished by waste producers, such as, for example, residential households, restaurant facilities, factories, other businesses, schools and the like. Waste producers can be provided with two or more different bins into which waste items may be placed according to type. Alternatively, waste sorting can be performed by personnel at waste sorting facilities and/or the like.

As used herein, the term “solid waste” is a broad term and may include, without limitation, municipal waste, industrial waste, sludge, fertilizer, manure, dairy waste, food waste, high moisture solid waste, low moisture solid waste, slurry and/or the like. In addition, for convenience, many of the embodiments disclosed herein refer specifically to a composting apparatus. It will be appreciated, however, that such apparatuses can also be used to dewater the materials situated therein, either in lieu of or in addition to composting.

Further, as used herein, the term “digester” is a broad term and is used in accordance with its ordinary meaning and may include, without limitation, any vessel, tank or other apparatus that is configured to dewater and/or treat, whether biologically (e.g., aerobic degradation, anaerobic degradation, anoxic degradation, etc.) or non-biologically, a volume of solids, liquids and/or other materials placed therein. The terms “composting apparatus,” “composter,” “digester,” “dewatering/composting apparatus” and “digestion apparatus” are used interchangeably herein.

As a result of such sorting methods and systems, recyclable 106, green 104 and/or other waste types 108 can be handled differently from the remaining solid waste volume. For example, recyclables 106 may be sent to a recycling center while green waste 104 can be used as landfill cover, for other land application purposes and/or the like. Moreover, it may be desirable to dispose of certain types of sorted or unsorted waste directly to a landfill 110.

With continued referenced to FIG. 1, unsorted waste, such as, for example, waste materials placed in non-compartmentalized municipal waste bins, can then be directed to a screening step 112. As described below, screening 112 can comprise a trommel screen, hand sorting and/or other devices or methods that further separate the waste into two or more different categories (e.g., type, size, etc.). For example, unsorted waste can include a compostable waste portion, which may undergo composting during a subsequent composting step 118, and another waste portion, which may be include landfillable materials 110, recyclables 106, larger paper products 114 (e.g., cardboard, paper packaging, etc.) and/or other waste materials 108. As indicated in FIG. 1, following composting 118, the treated compost 120 may undergo further treatment, such as, for example, screening, dewatering and/or the like 130. This is merely only one embodiment of a solid waste treatment method or scheme. As such, it will be appreciated that treatment methods or systems can include different and/or more or fewer treatment steps or processes.

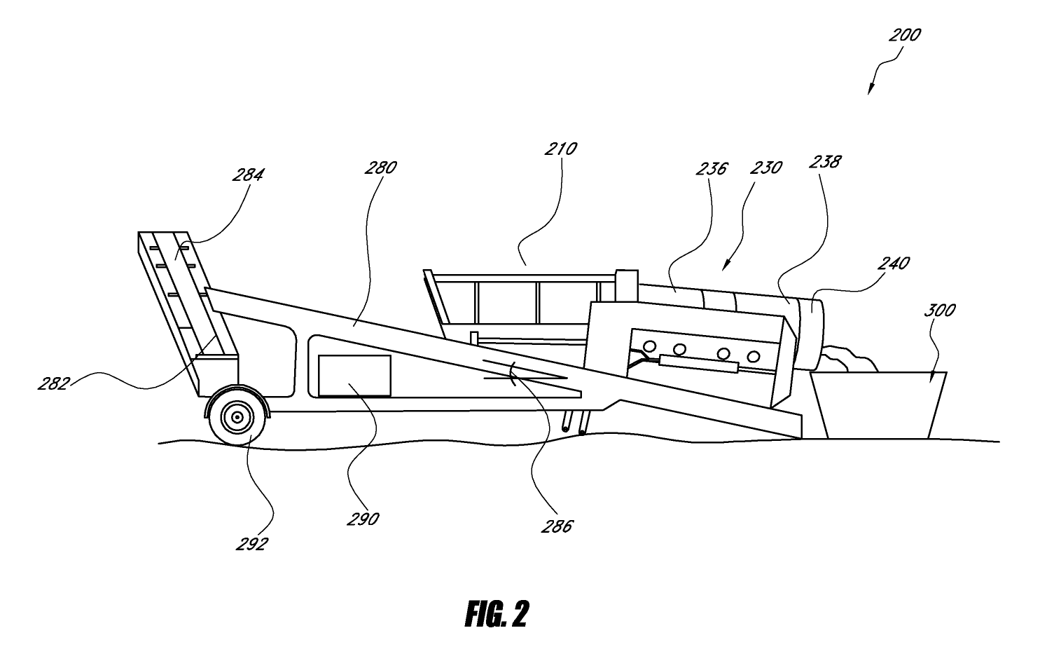

FIG. 2 illustrates a trommel screen 200 configured for placement upstream of a composting unit (not shown). The trommel screen 200 can comprise an inlet hopper 210, a screening drum 230, one or more conveyor systems 280, 284 and/or one or more other features. As described herein, waste material placed within the inlet hopper 210 can move into the interior of the rotating screening drum 230. Materials passing through the openings of the screening drum 230 can be configured to fall onto a conveyor 280 or other collection system, which can then transfer the screened material away from the screening drum 230. As shown in the illustrated embodiment, a conveyor 280 that is positioned underneath the screening drum 230 can be inclined at a particular angle 286 relative to horizontal so that the screened material can be simultaneously moved upwardly my the moving conveyor.

With continued reference to FIG. 2, the screening system can include a second conveyor 284 configured to receive the waste material carried by the first conveyor 280. This can permit screened waste to be moved toward a different direction and/or to another general area. A chute 282 or other channeling device can be used to facilitate the transfer from the first conveyor 280 to the second conveyor 284. Alternatively, one or more other methods or apparatuses can be used to move the screened waste from one location to another. For example, the system can be operated under a batch mode in which screened waste is collected in a bin and subsequently moved to a desired location by a truck or other vehicle. It will be appreciated that any other method or device for moving waste can be used.

As described herein and illustrated in FIG. 2, waste materials that do not pass through the openings of the screening drum 230 can move to the outlet end of the screening drum 230, where they may be deposited into a bin 300 or other container. According to some embodiments, this fraction of waste can be landfilled or subjected to additional sorting and/or treatment. For example, larger cardboard items, similar paper products or other compostable materials may be separated for later placement into a composting unit. In addition, glass, aluminum, plastic and/or other materials can be separated for recycling purposes.

In some embodiments, the trommel screen 200 includes one or more motors 290 which can be used to operate the conveyors 280, 284, 216, to rotate the screening drum 230 and/or drive one or more other mechanical and/or electrical components. Further, the trommel screen 200 can include wheel assemblies 292 or the like that facilitate in its transportation from one location to another.

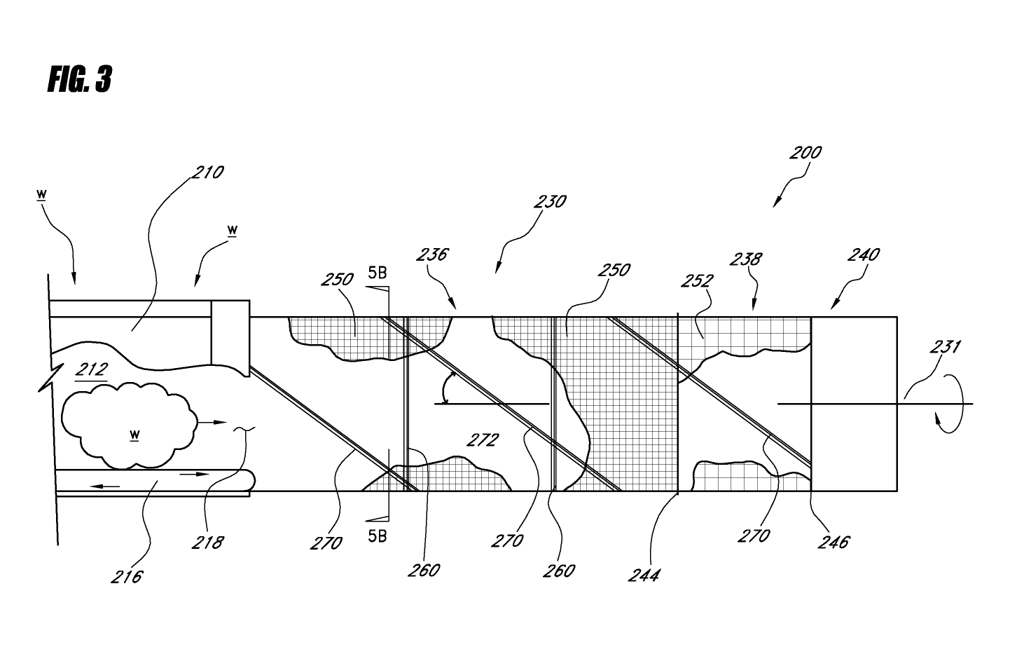

FIG. 3 illustrates a side view of the inlet hopper 210 and the screening drum 230 of the trommel screen 200 shown in FIG. 2. As depicted, in some embodiments, the bottom of the inlet hopper 210 comprises an inlet hopper conveyor 216 that can be configured to deliver waste items W from the inlet hopper 210 toward the screening drum 230 (in a direction from left to right as shown in FIG. 3). Waste items can be deposited directly into an interior portion 212 of the inlet hopper 210 by a truck or other waste collection vehicle (not shown). Alternatively, the waste items can be first processed at a sorting station or other facility before being placed into the trommel screen 200. In some embodiments, one or more mechanical devices (not shown) can be used to facilitate the placement of waste items into the inlet hopper 210. For example, a conveyor belt, an elevator, a vehicle equipped with a lifting member (e.g., bulldozer, front loader, etc.) or other lifting device can be used to deliver waste items into the inlet hopper 210.

In order to reduce the likelihood that glass and/or other impact-sensitive materials contained within the solid waste will break, one or more interior portions of the inlet hopper interior 212 can be cushioned. In one embodiment, one or more interior surfaces of the inlet hopper 210 can include a foam pad, a coating (e.g., polyurethane, elastomeric, etc.) and/or the like. For example, the inlet hopper conveyor 216 can be configured to provide the necessary cushion and/or flexibility in order to reduce the likelihood that glass and other materials will break upon placement in the inlet hopper 210. In one arrangement, the conveyor 216 can include a thick rubber layer and/or can be configured to resiliently move downward in response to a vertical load. The location, thickness, durability, resiliency and other properties of the pad, coating or other protective member can be advantageously selected to reduce the possibility of breakage or other undesirable damage.

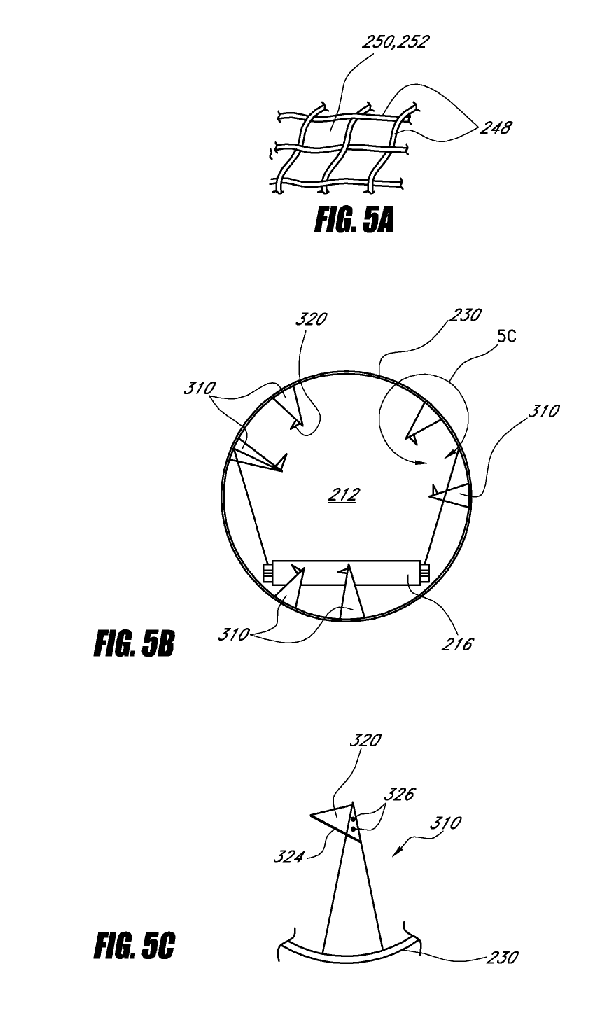

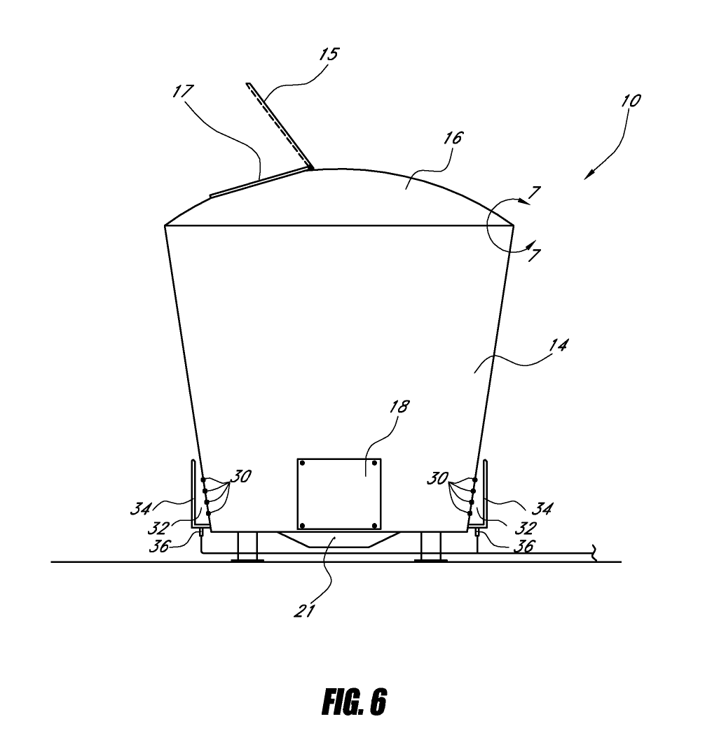

With continued reference to FIG. 3, the inlet hopper conveyor 216 can be configured to transfer waste items from the inlet hopper 210 to the interior of the screening drum 230. In the depicted arrangement, the waste items enter the interior of the screening drum 230 through an outlet 218 of the inlet hopper 210. The surface of the screening drum 230 can include a plurality of openings through which certain waste items may pass. The screen openings of the screening drum 230 can be created or formed using one or more suitable methods. For example, as illustrated in FIG. 5A, interwoven metal mesh 248 or wire can be shaped into the desired cylindrical shape. Alternatively, the screening drum 230 can comprise holes, perforations or other openings.

The size of the openings can be chosen according to the particular application and/or desired screening size range. In some embodiments, the size of the openings remains consistent throughout the entire length of the screening drum 230. Alternatively, the size of the openings can vary. In other embodiments, one or more portions of screening drum 230 need not have openings at all.

In the embodiment illustrated in FIG. 3, the size of the openings 250, 252 vary along the length of the screening drum 230. For example, in a first section 236 of the screening drum 230, the screen openings 250 can be approximately 2 inches by 2 inches. In contrast, in a second, downstream portion 238 of the drum 230, the screen openings 252 can be larger, such as, for example, approximately 4 inches by 4 inches. It will be appreciated that the screen opening sizes can be larger or smaller than disclosed and illustrated herein. In addition, the change in opening size along the length of the drum 230 can also vary.

A varying screen opening size along the length of the screening drum 230 can further facilitate sorting of waste. For example, in FIG. 3, smaller waste materials can pass through the screen openings 250 during an upstream portion 236 of the drum 230. As discussed, this fraction of the waste can be collected by a conveyor 280 (FIG. 2) and transferred to subsequent handling and/or treatment steps. Consequently, larger waste materials can remain within the interior of the drum 230 and move toward the drum outlet 231.

Depending on the type of waste being sorted, the size of the openings 250, 252, subsequent sorting or treatment methods and/or one or more other considerations, it may be desirable to place bins or other containers (not shown) underneath one or more locations of the drum 230. Thus, waste items passing through the larger openings 252 can be advantageously collected and separated from the smaller waste items that ultimately fall onto the conveyor 280 (FIG. 2). The screening drum 230 can comprise additional areas or zones with openings of varying sizes to further facilitate and enhance the sorting of waste.

As illustrated in FIG. 3, the drum 230 can include a solid section 240 that lacks openings. Alternatively, such a section 240 may comprise small perforations or other openings that permit primarily only liquids (e.g., water, leachate, etc.) to pass through the drum 240. In some embodiments, larger waste items retained within the screening drum 230 can be hand sorted into various categories (e.g., recyclable, aluminum, glass, paper, etc.) at or near this solid section 240. In other embodiments, as illustrated in FIG. 2, such large waste items can be discharged onto a conveyor or into a bin 300 or another container positioned beneath the outlet 231 of the screening drum 230. Waste captured in such bins or containers can be further sorted or treated, as desired. For example, as discussed further herein, cardboard and other paper-based products capable of being composted can be separated and subsequently fed into one or more shredders, composting units and/or the like.

With continued reference to FIG. 3, one or more flanged joints 244, 246 or other types of connections can be used to join the different sections of the screening drum 230. Other attachment methods can also be used, either in lieu of or in addition to flanged connections. For example, drum sections can be attached using welds, fasteners, slip fittings, threads and/or the like. In other embodiments, the screening drum 230 can be a unitary member that may or may not include variations in screen size along its length.

As illustrated in FIG. 2, the screening drum 230 can be sloped to facilitate the movement of larger items retained within the interior of the drum 230 towards the drum outlet 231. The movement of the waste items through the drum 230 may also be aided by the rotation of the drum 230. In some embodiments, the slope and/or the rotational speed of the screening drum 230 can be easily varied as required or desired by a particular user.

In order to strengthen its structural integrity, the screening drum 230 can include one or more reinforcing ribs 260 or other structural members. In FIG. 3, the illustrated trommel screen 200 comprises two circumferential reinforcing ribs 260 along the drum 230. However, it will be appreciated that more or fewer reinforcing ribs 260 can be used on a particular drum 230. In addition, the reinforcing ribs 260 can have a different position, size, shape, orientation and/or general configuration than shown in FIG. 3. For example, in some embodiments, the ribs 260 can be generally circumferential, longitudinal or diagonal (e.g., any angle in between longitudinal or diagonal).

In the depicted embodiment, the reinforcing ribs 260 can be flat metal bars that are shaped to match or substantially match the shape of the inner or outer diameter of the screening drum 230. The bars can be welded, fastened or otherwise attached to the inside and/or the outside surface of the drum 230.

One or more factors can be considered in determining whether or not reinforcing ribs 260 or other structural members are needed or desired, such as, for example, the diameter, length, thickness, materials of construction and other characteristics of the screening drum 230, the anticipated size, volume, weight and other characteristics of the waste entering the drum 230, the anticipated rotational speed of the drum 230 and the like.

With continued reference to FIG. 3, the screening drum 230 can comprise one or more internal channeling members 270 that are configured to help reduce the likelihood that glass and other impact-sensitive waste items will break during operation of the drum 230. The channeling members 270 can include rigid or semi-rigid flat bars or the like (e.g., manufactured from steel, iron, other metals, polymeric materials, other composites, etc.) that are positioned along an interior portion of the screening drum 230. In one embodiment, the width and thickness of the bars can be approximately 1-2 inches and ¼ inches, respectively. However, in other embodiments, the width and/or thickness of the bar can be smaller or larger, as desired or required by a particular application.

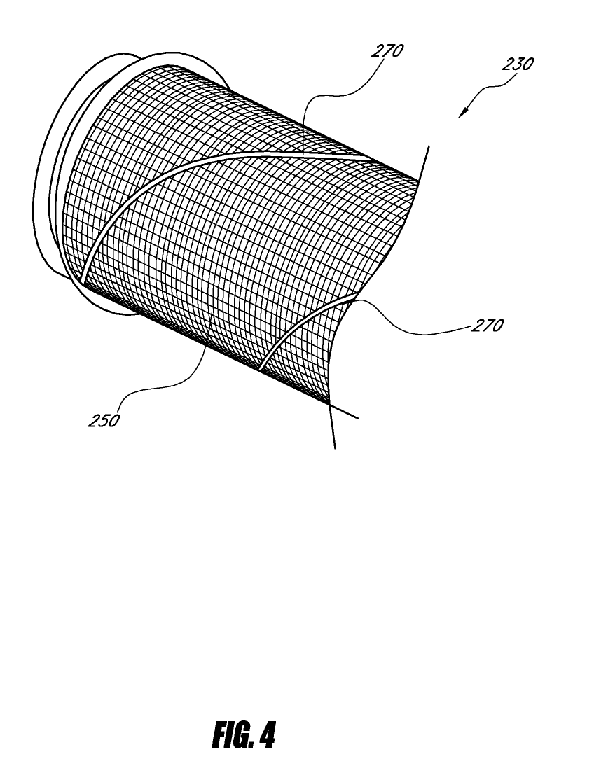

The channeling members 270 can be shaped and otherwise configured to generally match the internal surface of the drum 230. As illustrated in FIGS. 3 and 4, the channeling members 270 can be routed along the interior of the drum 230 in a spiral fashion, such that the position of the channeling members 270 relative to the drum's circumference changes along the length of the screening drum 230. Thus, in such arrangements, the channeling members 270 are positioned at an angle 272 relative to the longitudinal axis of the drum 230. One or more suitable connection devices and/or methods can be used to attach the channeling members 270 to the drum 230, such as, for example, welds, fasteners, adhesives or the like.

The channeling members 270 can include any other type of rigid or semi-rigid article. In addition, more or fewer channeling members 270 can be used in a particular screening drum 230. Further, the number, size, shape, dimensions, angle 272, connection methods and other characteristics of the channeling members 270 can be different than that disclosed or illustrated herein.

As discussed, the channeling members 270 can help prevent glass and other impact-sensitive waste items from breaking by causing the waste items to move in a generally smoother manner through and within an interior portion of the screening drum 230. For example, the channeling members 270 may lessen the frequency that glass items positioned along an upper portion of the interior of the drum 230 abruptly fall to a lower portion of the drum 230. In one embodiment, the channeling members 270 can allow waste items to move in a more methodical manner along the length of the drum so as to reduce the impact between different waste items and/or between waste items and the surfaces of the drum 230.

In lieu of or in addition to the use of channeling members 270, the interior surface of the screening drum 230, which the waste items contact, can include one or more layers of a protective coating. The coating can help reduce the likelihood that glass and other impact-sensitive waste materials will break when they impact the surface of the screening drum 230. The coating can include one or more layers of polyurethane, enamel, epoxy, plastic, paint and/or the like. The thickness of the coating can be varied depending on the particular application. For example, in some embodiments, the coating can be between 1/32 inch to ¼ inch thick. However, the thickness of the coating can be larger or smaller than this range, as desired or required by a particular situation. In addition, such coatings can advantageously extend the effective life of screening drums 230 by providing anti-corrosion, anti-abrasion and other benefits.

As illustrated in the cross sectional view of FIG. 5B, the screening drum 230 can comprise one or more bag rippers 310 or similar members. As shown, in some embodiments, the bag rippers 310 can be positioned near the outlet 218 of the inlet hopper 210. In the depicted arrangement, the drum 230 includes a total of six bag rippers 310. However, the drum 230 can comprise more or fewer bag rippers 310. The bag rippers 310 can have the same size, shape, dimensions and general configuration. Alternatively, the bag rippers 310 can have one or more characteristics (e.g., shape, size, position, etc.) that distinguish them from each other. For example, as illustrated in FIG. 5B, some of the rippers 310 can be larger than others.

The bag rippers 310 can be positioned along the same or different longitudinal location of the screening drum 230. In addition, as shown herein, the bag rippers 310 can be situated along different circumferential locations of the drum 230. Regardless, it may be desirable to include one or more bag rippers 310 immediately downstream of the inlet hopper outlet 218 (FIG. 3) so that trash bags (e.g., plastic, paper, etc.) and/or other containers can be punctured to advantageously release the waste contents contained therein onto the screening drum 230. This ripping process can be further facilitated by the rotational movement of the screening drum 230 relative to the inlet hopper 210.

The bag rippers 310 can be manufactured from one or more rigid and durable materials, such as, for example, steel, iron, other metals, composites and the like. The bag rippers 310 can be securely attached to the adjacent screening drum 230 using one more connection methods or devices, such as, for example, welds, fasteners and/or the like. Further, the bag rippers 310 can be permanently or removably attached to the drum 230. Removable rippers 310 can advantageously permit a user to replace dull or otherwise damaged bag rippers 310.

As illustrated in the detailed view of FIG. 5C, one or more of the bag rippers 310 can include a blade 320 along their upper portion. In some embodiments, the blade 320 comprises one or more sharpened surfaces 324 that facilitate the grabbing, ripping and/or cutting of the bags or other containers placed within the drum 230. In the depicted arrangement, the blade 320 is attached to the bag ripper 310 using two removable fasteners 326 (e.g., screws, rivets, etc.). This can facilitate the replacement of the blades 320 as they blunt, break, bend or otherwise become damaged. Other removable and/or fixed connection methods can also be used to attach the blade 320 to the rippers 310.

With continued reference to FIG. 5B, the bag rippers 310 can be oriented so that their blades 320 face in different directions. Alternatively, the bag rippers 310 can be positioned so that the blades 320 are similarly configured. In addition, a bag ripper 310 may include two or more blades 320 to further facilitate the grabbing or cutting action. The shape, size and/or other characteristics of the blades 320, as well as the position of the blades 320 relative to the bag rippers 310, can be different that discussed and illustrated herein.

The discussion below and the figures referenced herein describe various embodiments of an apparatus and method for composting and/or dewatering waste (e.g., organic waste, inorganic waste, municipal waste, etc.). A number of these embodiments are particularly well suited for implementation in a vertical in-vessel composting unit. However, it will be appreciated that the features and advantages related to the different embodiments described herein may be incorporated into other types of composting and/or dewatering units, including horizontal composting units, non-enclosed composting units and/or the like.

The embodiments described herein can help enhance the composting of organic and other biodegradable materials. Consequently, by implementing such apparatuses and methods, or variations thereof, a higher quality and more consistent compost can be produced within a shorter time period. In addition, significant environmental benefits can be achieved because the total volume of waste directed into volume-limited landfills and other similar disposal sites can be reduced. In some embodiments, the nutrient-rich compost can be land applied and/or used in other beneficial or non-beneficial applications.

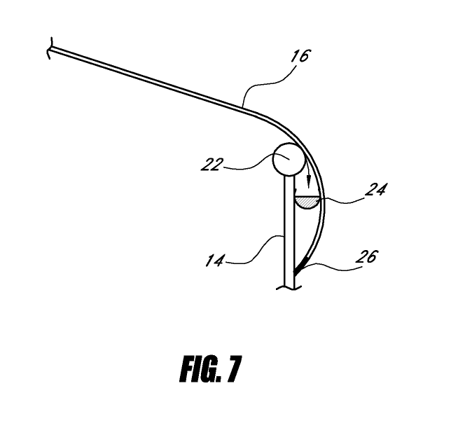

FIG. 6 illustrates an in-vessel composting unit 10 comprising a main vessel portion 14 and a cover 16. The depicted vessel portion 14 has generally rounded walls that taper inwardly toward the bottom of the composting unit 10. In some embodiments, the vessel portion 14 can comprise a generally cylindrical (e.g., vertical walls, wall being angled relative to vertical, etc.), frusto-conical, curved and/or the like. This applies to all embodiments of composting/dewatering units disclosed and illustrated herein. In the illustrated embodiment, the composting unit 10 includes a sediment hopper 21 which is preferably positioned at a low point within the interior of the vessel portion 14. The sediment hopper 21, which is configured to receive sand, grit and/or other coarse materials, can include a drain or cleanout (not shown) to facilitate emptying. In one embodiment, the vessel portion 14 comprises one or more rigid materials capable of withstanding corrosion, abrasion, environmental conditions (e.g., rain, sunlight, etc.), fluctuations in temperature, pH, liquid level, internal pressure fluctuations and/or the like. For example, the vessel portion 14 and/or other parts of the composting unit 10 may comprise steel, iron, concrete, fiberglass and/or any other suitable material. In addition, the composting unit 10 can be advantageously designed to resist wind, earthquakes and other forces that may act upon it.

In some embodiments, the vessel portion 14 can have a different general shape than disclosed or illustrated herein. For instance, the shape of the vessel portion 14 can be generally rectangular, egg-shaped, cylindrical and/or the like. Further, the taper of the vessel's outer wall can be different than illustrated herein. For example, the taper angle can be greater or less than illustrated and/or can include a different shape or orientation. In one embodiment, the walls of the vessel portion 14 are substantially vertical. Further, the composting unit 10 can include wheels (not shown) or may be configured to be positioned on a movable member (e.g., flatbed truck, trailer, etc.) for easy transport or relocation.

With continued reference to FIG. 6, the vessel portion 14 can include one or more hatches 18 or accessways. In FIG. 6, a rectangular shaped hatch 18 is located near the bottom of vessel portion 10, close to the sediment hopper 21. The hatch 18 can be advantageously removed to provide access to the inside of the composting unit 10 for cleaning, maintenance and/or any other purpose. The hatch 18 or other opening can be sized for easy ingress into and egress out of the interior of the composting unit 10. Further, the hatch 18 can be connected to the wall of the vessel portion 14 using one or more attachment methods or devices, such as, for example, bolts, hinges, slide railings, flanges, other fasteners and/or the like. In addition, a gasket or other compressible member can be positioned between the hatch 18 and the vessel portion 14 to provide a better seal.

In FIG. 6, the composting unit 10 includes a generally dome-shaped cover 16 that extends around the entire periphery of the vessel portion 14. However, the cover 16 can have any other shape, such as, for example, flat, conical, concave, convex, frusto-conical, frusto-spherical and/or the like. The cover 16 can be constructed of one or more rigid, semi-rigid and/or flexible materials. In one embodiment, the cover 16 comprises a generally durable thermoplastic material which is specially sized, shaped and otherwise configured to fit over the vessel portion 14. In other embodiments, the cover 16 can simply include a sheet, tarp, fabric or other material that is stretched around the top opening of the vessel portion 14.

The cover 16 can be manufactured from plastic, rubber, tarp, metal and/or any other materials. The composting unit 10 can comprise a frame (not shown) or other similar system to generally support the cover 16. This can be especially useful if a flexible or semi-rigid cover 16 is used. It will be appreciated that the frame can include one or more rigid members that span either partially or completely across the upper opening of the vessel portion 14. For example, the frame can include a steel truss that is supported by the vessel portion 14 and helps maintain the cover 16 in a desired shape or orientation.

In one arrangement, the inside surface of the cover 16 can be roughened and/or can to include a layer having a generally high surface area. For example, a layer of artificial turf or other material can be placed underneath the cover 16. The layer can be attached to the cover 16 using adhesives, fasteners and/or any other connection method or device.

A relatively high surface area layer can provide a medium on which bacteria and other microorganisms can grow. In some embodiments, such microorganisms can become acclimated to consume or otherwise eliminate certain problematic compounds that may otherwise accumulate on the bottom surface of the cover 16. For example, sulfur gas released during the decomposition of the waste materials may form sulfuric acid on the bottom of the cover 16. Such acidic materials can cause the cover 16, vessel portion 14 and other components of the composting unit 10 to corrode, deteriorate or otherwise become damaged, especially over time. Thus, techniques that promote the presence of bacteria which are able to consume, biodegrade or otherwise alter these problematic compounds can be utilized to advantageously eliminate or reduce damage to the composting unit 10. The roughened surface or high surface area layer (e.g., artificial turf) can promote the growth of biofilms or other colonies of such desirable microorganisms.

With continued reference to FIG. 6, the cover 16 can include one or more cover openings 17. In some arrangements, a cover opening 17 can provide access to the interior of the composting unit 10 for feeding, venting, aeration, maintenance and/or the like. In the depicted embodiment, a lid member 15 is hingably attached to the cover 16 to selectively cover or expose the adjacent cover opening 17. However, in other embodiments, the lid member 15 can be attached to the cover 16 using a different type of connection method or device (e.g., sliding door or accessway). Alternatively, the lid member 15 may need to be completely separated from the cover 16 in order to expose the cover opening 17. Preferably, the cover opening 17 is shaped, sized, positioned and oriented to facilitate with the feeding of the composting unit 10 and/or any other activities.

FIG. 7 illustrates one embodiment of the interface between the cover 16 and the vessel portion 14 of the composting unit 10. As shown, the cover 16 can be positioned over a support, such as a spherical member 22, located at or near the upper end of the wall of the vessel portion 14. It will be appreciated that any other suitable support structure and/or shape can be used. In FIG. 7, the cover 16 is attached to an exterior area 26 of the vessel portion 14 using one or more connection methods or devices.

With continued reference to FIG. 7, a condensation collection member 24 can be situated in the gap 23 defined by the exterior of the vessel portion 14 and the interior of the cover 16. In some embodiments, such a liquid collection member 24 can be configured to collect condensation and other liquids that collect on the cover 16 and flow (e.g., drip) downwardly along an interior surface of the cover 16. Thus, one or more channels, grooves and/or other openings can be advantageously positioned between the spherical member 22 and the cover 14 to allow any liquid moving along the inside of the cover to enter the gap 23. Water and other liquid collected by the condensation collection member 24 can be advantageously removed from the composting unit 10 using one or more pipes or other conveying members. The condensation collection member 24 can be positioned entirely or partially around the cover-vessel portion interface.

In one embodiment, the cover depicted in FIG. 7 comprises a flexible plastic sheet that stretches over the spherical member 22 and the condensation collection member 24 before it attaches to the vessel portion 14. The sheet can attach to the vessel portion wall using one or more snap fit connections, hooks, clamps and/or using any other device or method. In some embodiments, the spherical member 22 and/or the condensation collection member 24 comprise polyvinyl chloride (PVC), plastic, steel, copper, iron, other metals, composites and/or any other suitable material. Alternatively, the composting unit 10 does not need to include a condensation collection member 24 at all, allowing the liquid to simply roll down the outside surface of the vessel portion 14. One or more other collection members or methods can be incorporated into the design of the composting unit 10 for the removal of condensation and/or other liquids.

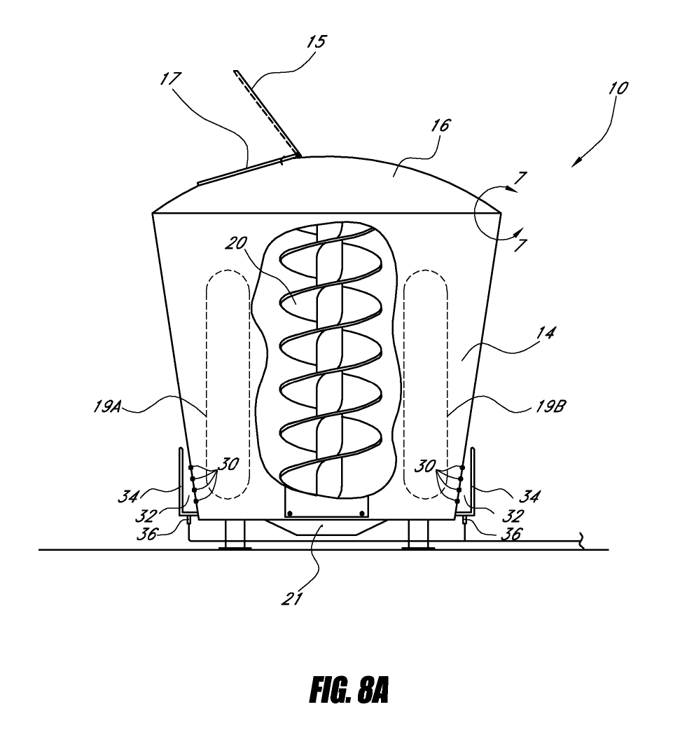

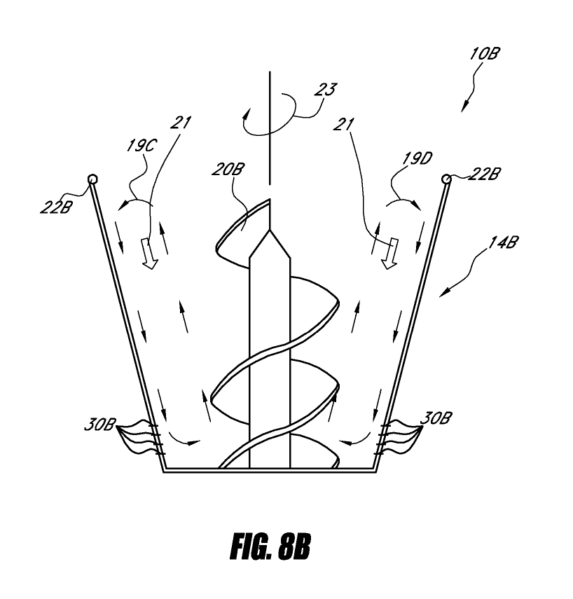

FIG. 8A illustrates a composting unit 10 similar to the one shown in FIG. 6 with a section of the vessel portion 14 removed to reveal an interior area. As shown, the interior of the composting unit 10 can comprise one or more augers 20 or other mixing members to allow the materials being processed to be selectively mixed or agitated. As will be discussed in greater detail below, the auger 20 can also facilitate dewatering of waste while the composting unit 10 is being operated.

In the illustrated embodiment, a single auger 20 is positioned near the center of the composting unit 10. The auger 20 is supported at or near the bottom floor of the vessel portion 14 and extends vertically toward the cover 16. However, the position and orientation of the auger 20 within the composting unit 10 can be different than discussed and illustrated herein. In one embodiment, in order to enhance mixing within the composting unit 10, the top of the auger 20 is positioned at or near the top of the vessel portion 14. Alternatively, the auger 20 may extend to about halfway or more than halfway the height of the vessel portion 14. For example, the top of the auger 20 can be at or near three-fourths the total height of the vessel portion 14. However, in other embodiments, the auger 20 may extend below the halfway height of the vessel portion 14.

Depending on the dimensions, operating conditions (e.g., temperature, moisture content, etc.), waste stream characteristics and/or one or more other factors affecting the composting process, two or more augers 20 can be included within a single composting unit 10. Augers 20 can have a vertical, horizontal, diagonal or any other orientation. If a composting unit 10 includes two or more augers 20, the augers 20 can be parallel and/or non-parallel to each other. In addition, augers 20 within the same composting unit 10 can vary from one another with respect to length, shape and/or any other characteristics. Regardless of their exact size, dimensions, shape, positioning and location within the composting unit 10, the augers 20 can be advantageously configured to rotate about a longitudinal axis. As is discussed in greater detail herein, this rotating motion can facilitate the mixing and/or dewatering of the waste materials being treated within the unit 10. Preferably, the augers 20 are configured so that their rate of rotation can be modulated (e.g., increased, decreased), allowing operators to modify the composting operation in response to one or more factors or as otherwise desired.

FIG. 8B illustrates another embodiment of a composting unit 10B comprising an auger 20B that is generally cone-shaped. As shown, the auger 20B includes fewer threads than the auger depicted in FIG. 8A. However, in other embodiments, the auger 20B can include more or fewer threads than illustrated in FIG. 8A or 8B.

In FIG. 8B, the auger 20B is configured to rotate in a clockwise direction when viewed from the top, as indicated by the arrow 23. Rotation of the auger 20B can cause the internal contents of the composting unit 10B to move in a generally circular manner as illustrated by arrows 19C, 19D. This can help maintain the waste materials situated within the composting unit 10B well-mixed. Further, the downward movement of the solids next to the openings 30B along the lower end of the vessel portion 14B can facilitate dewatering of the solids. The dewatering also can be aided by the geometry of the vessel 14B and/or the orientation of the auger 20B relative to the walls of the vessel 14B, as compression zones can be created at or near the bottom of the composting unit 10B (e.g., near the openings 30B). In addition, the downward force created by the head pressure (represented by arrows 21) of the solids contained within the composting unit 10B can further enhance dewatering through the openings 30B.

Although not shown in the illustrated embodiments herein, the composting unit 10 can optionally comprise an aeration system for providing air into the interior of the unit 10 during operation. Generally, some microbes or other microorganisms responsible for the composting process require oxygen to adequately and efficiently decompose the waste materials fed into the composting unit 10. Thus, air or other fluid containing oxygen (e.g., pure oxygen) can be advantageously piped into the interior of the composting unit 10 to ensure that the composting process is not negatively affected. One or more blowers, air compressors or other fluid transfer devices can be used to deliver a desired air flow within one or more regions of the composting unit 10. The air or other fluid can be conveyed to one or more discharge points through a piping system. In one embodiment, air diffusers or similar air distribution members may be used to dispense air throughout an interior portion of the composting unit 10.

Alternatively, the interior of the composting unit 10 can be in fluid communication with the ambient environment during the composting process, such as, for example, by removing the cover or a portion of it (e.g., the cover's lid member 15 in FIGS. 6 and 8A). This can permit air to enter the interior of the composting unit 10 and at least partially aerate the material (e.g., waste) contained therein. Depending on the oxygen demand required for composting, it may be necessary to supplement the process by using a blower, compressor or other fluid transfer device to provide additional air or other fluid into the composting unit 10. In other embodiments, the composting can be operated under limited oxygen or no oxygen (e.g., anoxic, anaerobic, etc.). Such a change can affect the manner in which the composting unit is operated and/or the characteristics of the composed materials.

Further, heating and/or cooling devices can also be included to generally control the temperature within the interior of the composting unit 10. Generally, microbes and other microorganisms are most efficient when the surrounding temperature is within a particular target temperature range. For example, certain microorganisms prefer relatively cold environments, such as, for example, environments where temperatures are near or below 25° C. Other microorganisms are most active in relatively moderate temperature ranges, such as, for example, temperatures between 20° C. and 45° C. Yet other microorganisms exhibit optimum growth rates in relatively warm environments, such as, for example, environments where temperatures are near or above 45° C. or greater. Typically, heat is a by-product of the composting process. Therefore, the temperature inside the composting unit 10 tends to rise as the composting process develops. However, in order to optimize the composting process, it may be desirable to increase or decrease the internal temperature of the composting unit 10. Temperature control devices can include heaters, heating/cooling channels, heat exchangers and the like. In order to monitor and/or control such an internal temperature, the composting unit 10 can comprise temperature sensors, control units and/or other components.

With continued reference to FIGS. 6 and 8A, the vessel portion 14 can comprise a plurality of openings 30 along one or more of its walls. In the illustrated embodiments, the openings 30, which are in fluid communication with the inside of the composting unit 10, are located near the bottom of the vessel portion 14. The number, shape, size, location, spacing and/or other characteristics of the openings 30 can be customized to the particular composting procedure being performed. In one embodiment, the openings 30, which are configured to discharge water and other liquids contained within the vessel portion 14, are circular and have a diameter of approximately ½ inch. In addition, the openings 30 may be configured to permit air to enter into and/or exit from the interior of the vessel portion 14 under certain desired conditions. The openings 30 can be smaller than ½ inch, such as for example, ⅛ inch, 1/16 inch, 1/32 inch or smaller, or ranges encompassing such values. Alternatively, the openings 30 can be larger than ½ inch, such as for example, ¾ inch, 1 inch, 1½ inch, 2 inch or greater, or ranges encompassing such values.

In some embodiments, the vessel portion 14 may comprise openings 30 having two or more different shapes, sizes or other characteristics. The openings 30 can be shaped, sized, positioned, spaced and/or otherwise configured according to the type of waste material that will be placed within the composting unit 10 or as otherwise is desired by the user. For example, it may be desirable to include relatively smaller openings if biosolids, primary sludge, secondary sludge, digested sludge or other materials generated by a wastewater treatment facility are to be treated. This can help reduce or prevent the undesirable movement of waste materials through the openings 30.

As illustrated in FIGS. 6 and 8A, water or other liquids exiting the vessel portion 14 through the openings 30 can be collected in a collection area 32. Such a collection area 32 can be defined by a collection structure 34 positioned on the outside of a vessel portion wall, on the inside of a vessel portion wall and/or embedded in the wall of the composting unit. In the illustrated embodiments, the collection structure 34 comprises a box-like member formed by one or more rigid or semi-rigid members. For example, the collection structure 34 can be formed by welding two or more steel plates to one another. The collection structure 34 may be advantageously shaped to generally match or otherwise complement the exterior shape of the vessel portion 14 to which it attaches.

The collection structure 34 can be permanently or temporarily attached to the vessel portion. For example, the collection structure 34 can be removably attached to the vessel portion 14 using one or more connection methods or devices, such as, bolts, slide fittings, clips, clamps, other fasteners and/or the like. In other embodiments, the collection structure 34 may be formed from the same unitary body as the composting unit 10 (e.g., using welds or the like).

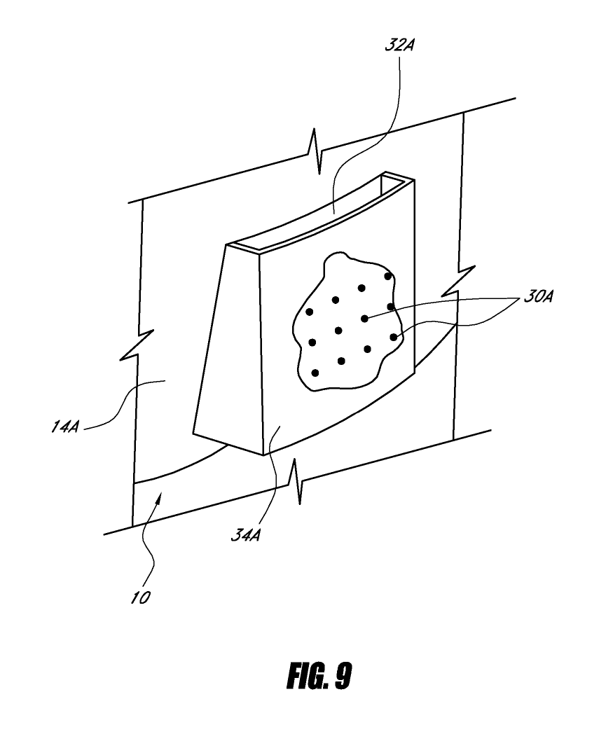

FIG. 9 illustrates another embodiment of a collection structure 34A positioned along the outside of a vessel portion 14A. In FIG. 9, a portion of the collection structure 34A is removed to reveal a number of openings 30A on the vessel portion 14A. Regardless of its shape, location, configuration, dimensions and/or other characteristics, the collection structure 34A preferably defines a collection area 32A into which the openings 30 will discharge. Thus, liquid exiting a composting unit 10 can be captured and collected within the collection area 32A.

As depicted in FIG. 8A, the collection structure 34 can comprise a drain 36 or other outlet which allows the collected liquid to be easily removed. Depending on one or more factors, such as the size of the composting unit 10, the liquid content and general composition of the material being composted and/or the like, a composting unit 10 can comprise one, two or more collection structures 34. For example, in some embodiments, a vessel portion 14 includes four collection structures 34, each of which is advantageously spaced to provide more efficient removal of water and other liquids from the adjacent composting unit 10.

In one arrangement, the collection structure 34 is completely or partially sealed off from the atmosphere. This may be significant for odor control purposes, especially if the liquid or other fluids discharged from the openings 30 are malodorous. Alternatively, the collection area 32 can be open or selectively openable (i.e., completely or partially sealable) to the surrounding environment. One or more openings 30 may be positioned on the underside of the vessel portion 14, either in lieu of or in addition to any openings 30 located on the sidewalls. Therefore, in order to collect the volume of liquid discharged from the composting unit 10, it may be desirable to include a collection structure or similar member along the bottom and/or in any other area of the composting unit 10 that comprises an opening.

As illustrated in FIG. 8A, liquid discharged into the collection structures 34 can be directed into an interconnected piping system 38. The piping system 38, which in some embodiments includes one or more fittings (e.g., tees, elbows, valves, etc.), can be configured to channel liquid to another location for disposal, treatment and/or further processing. The piping system 38 can comprise steel, PVC, plastic, copper, galvanized steel, iron, ductile iron, rubber and/or other suitable material. In one embodiment, as depicted in FIG. 10, liquid is transferred from a collection structure 34 to a clarifier tank 40 or another treatment process. The liquid can be configured to flow by gravity from a collection structure 34 to the clarifier tank 40 or other treatment step to reduce or eliminate the need for pumps or other mechanical devices. Alternatively, liquid from the collection structures can be discharged into a sewer, drain and/or the like.

With continued reference to the embodiment of FIG. 10, the clarifier tank 40 includes an inlet 51 through which liquid is discharged. It will be appreciated that a clarifier tank 40 can include additional inlets 51. In addition, the tank 40 can comprise a screen 42, membrane and/or other separation member that is used to remove solids from the liquid stream or otherwise treat the liquid being discharged into the tank 40. In one embodiment, a lower outlet 54 can be provided downstream of a screen 42, allowing the some or all of the solids contained in the liquid that passes through the screen 42 to be separated or removed.

Alternatively, the clarifier tank 40 may not include a screen 42 or other physical separation member. In such embodiments, liquid can be directed into the tank 40 and can be given sufficient time to naturally separate into one or more layers. For example, over time, the heavier solids contained within the liquid stream may settle towards the bottom of the clarifier tank 40. Further, grease, oil and other substances having a lower density may float toward the top of the liquid level. Consequently, a plurality of outlets 52, 53, 54 can be positioned along various heights of the tank 40 to permit selective removal of one or more different types of liquid or solids. In other embodiments, a series of such tanks 40 can be provided.

In the embodiment illustrated in FIG. 10, the clarifier tank 40 includes a total of three outlets 52, 53, 54 positioned at different elevations. The bottom outlet 54 can be used to remove sludge, solids or other heavier materials that have settled toward the bottom of the tank 40. An upper outlet 52 can be configured to remove oil, grease and other floatable materials. Further, the middle outlet 53 can be used to remove water and/or other liquids retained near the middle portion of the tank 40. It will be appreciated that more or fewer outlets may be included, and that the size, shape, spacing, location, general configuration and/or other details about the outlets can be different than discussed and illustrated herein.

Moreover, one or more other treatment features can be incorporated into the clarifier tank 40 design. For example, the tank 40 can comprise a mixer, aerator, chemical injectors, baffles and/or the like. The clarifier tank 40 can be operated either as a batch or a continuous system. Further, the operation of the tank 40, including filling, emptying, etc., can be automatic or manual. In some embodiments, the treated water can be re-introduced into the composting unit 10, especially at later stages of the composting process, in order to maintain a desired moisture level for the waste materials being treated therein. Alternatively, depending to the extent to which it is treated, liquid discharged from the tank 40 can be directed to a drain or another location. In some embodiments, the treated liquid may be used as washwater and/or for other non-portable purposes.

In certain embodiments, the clarifier tank 40 comprises one or more rigid or semi-rigid materials, such as, for example, plastic, steel, iron, aluminum, other metals or composite materials and/or the like. In addition, the size, shape, dimensions, capacity, location of inlets and outlets and other characteristics of the tank 40 may be varied, as desired or required by a particular user or application. Further, the clarifier tank 40 can also be configured to accept other liquid waste, such as, for example, condensation and/or other liquids directed into the condensation collection member 24 (FIG. 7).

The composting unit 10 can be operated as either a batch or a continuous system. Under a batch operation mode, organic materials and/or other waste items are fed into the composting unit 10 at the beginning of a composting cycle and are not removed until the composting cycle ends. It will be appreciated that additional items, such as liquid discharged from the openings, liquid collected in the clarifier tank, cardboard items and the like, may be added at later stages of a composting cycle. Typically, however, waste is not continuously added into or removed from the composting unit 10 under such an operational scheme. Conversely, the composting unit 10 can be operated under a continuous mode where waste material is constantly or intermittently fed into the composting unit 10, and composted waste is constantly or intermittently removed from it. This type of operational scheme can facilitate the processing of waste, as the need for storage of organic and other compostable waste material is reduced or eliminated. It will be appreciated that the various embodiments of the composting unit 10 and other ancillary systems described herein are equally applicable to batch and continuous type systems.

For convenience, one embodiment of the operation of the composting unit 10 will be described herein assuming that a batch type scheme is used. However, it will be appreciated that at least some of these operation steps and features can be applied to a continuous or other type of system. In one embodiment, a volume of organic material and/or other compostable waste is initially fed into the composting unit 10 through a cover opening 17 as depicted in FIG. 6. Alternatively, the cover 16 may be removed, either partially or completely, before initiating the feeding of the composting unit 10. In order to accelerate the decomposition process, it may be helpful to inoculate the composting unit 10 with a volume of active compost and/or any other source containing an active microbe mix. After the composting unit 10 has been adequately filled, the cover 16 can be placed over the vessel portion 14 to seal the unit 10. In embodiments where waste materials were fed into the composting unit 10 through one or more cover openings 17, any such openings can be selectively closed.

In some embodiments, one or more augers 20 located in the composting unit 10 begin operating as the composting unit 10 is being filled. Alternatively, the augers 20 may be initiated immediately after the filling phase has been completed or at some other time (e.g., after a prescribed time period following initiation of the composting process has elapsed). As discussed, in order to provide better operational control over the process, the rotational speed of each auger 20 can be adjustable. The augers 20 may be operated continuously or intermittently during the composting process.

Depending on the characteristics of the waste being composted, a volume of water and/or other liquid can be removed from the composting unit 10 through the plurality of openings 30 located on the vessel portion 14. Removal of water and/or other liquids from the waste materials, and thus the composting unit 10, can be facilitated by operation of an auger 20. In some embodiments, movement of the solids and other waste materials resulting from the operation of the augers can increase the amount of water and/or other liquids that are removed from a composting unit through the openings. Moreover, the weight of the material within the composting unit 10 can lead to additional dewatering through the openings 30. Since the composting process is more efficient when the water content of the materials within the unit 10 is within a desired range, it is often beneficial to initially remove a particular volume of water and/or other liquids. Alternatively, if the material being composted is too dry or becomes too dry during the composting cycle, water and/or other liquids may be added to the composting unit 10 to achieve a desired moisture level. As discussed, in some arrangements, water collected, and possibly treated, in a clarifier tank 40 (FIG. 10), can be returned to the composting unit to control the moisture level.

As the composting process continues, the temperature inside the composting unit 10 may naturally rise due to the increased microbial activity. In addition, the temperature can be artificially regulated (e.g., heated, cooled, etc.) using a heating and/or cooling device. Moreover, air or other oxygen-bearing fluids can be directed into the composting unit 10 to facilitate the microorganisms in their decomposition of the waste materials. In some embodiments, after the organic and other water materials being composted attain a particular temperature, additional water and/or other liquids can exit the composting unit 10 via the openings 30. This can result from one or more reasons, such as, for example, the physical, chemical, biochemical and/or other properties of the waste materials being treated, the fluid mechanics inside the vessel portion and/or the like.

In some embodiments, the augers 20 help to maintain the compostable materials well-mixed during the decomposition process. For example, as illustrated in FIG. 8A, operation of the auger 20 can cause the waste materials within the composting unit 10 to move in a pattern generally represented by arrows 19A and 19B. Of course, it will be appreciated, that the general movement of compostable materials in a composting unit 10 can be different than depicted in FIG. 8A. The mixing patterns and characteristics of a composting unit 10 may depend on one or more factors, such as, for example, the shape, size, dimensions, orientation or other features of the composting unit, including the augers, the viscosity, density, water content and other properties of the materials being processed and/or the like.

Under a batch operational scheme, the composting unit can be operated for a minimum time period before it is stopped. Preferably, this time period ensures that the waste materials are treated to a level that meets or exceeds the applicable regulatory and/or other applicable requirements. For example, an EPA or other regulatory agency requirements may necessitate that a minimum percentage of organic material decomposition to be attained before the composted materials can be land applied. Alternatively, a governing regulation can mandate that a particular time-temperature treatment option be achieved. In one embodiment, such regulations can be achieved by operating the composting unit 10 for approximately seven days under a mesophilic temperature range (e.g., between 0° and 45° C., or more preferably, between 20° to 35° C.). However, depending on the desired or required level of treatment, it will be appreciated that the compostable materials may need to be processed for longer or shorter time periods and/or at higher or lower temperatures than disclosed herein.

Once the composting process has been initiated, additional organic and/or other waste materials can be added to the composting unit 10. For example, cardboard or other large paper-based products can be fed into the composting unit 10 through one or more openings. Other organic or other waste materials can also be added into the composting unit 10, either in lieu of or in addition to paper-based waste, after the composting cycle has commenced. Under a continuous feed operational mode, waste materials can be fed into, and consequently can be removed from, the composting unit 10 at one or more regular intervals. The addition of organic materials while the composting unit 10 is operating can be used to control the carbon to nitrogen ratio, the food to microorganism ratio and other operational parameters or factors that can affect the composting process. Furthermore, as discussed, a volume of water or other liquid can be added to the composting unit 10 to maintain the moisture content within the unit 10 within a desired range. Polymers and/or other chemicals may also be added to further enhance the composting process.

The different embodiments of the composting unit described and/or illustrated herein can be used to treat different types of solid and/or liquid waste streams. For example, the composting unit can be used to treat municipal and/or industrial sludge (primary sludge, secondary/biological sludge, waste activated sludge, digested sludge, etc.). The composting unit can also be used to treat manure or other solid and/or liquid wastes originating from animal farms and the like.

After the waste materials have been adequately processed, the auger 20, air feed system and/or other systems or subsystems can be stopped. The treated compost can then be removed from the composting unit 10 for further processing or treatment, packaging, transport and/or the like. For example, the treated compost can be screened to remove larger items. In other embodiments, the treated compost can be dewatered, chemically or biologically treated and/or the like. The compost can be removed from the vessel portion 14 using a solids pump (e.g., non-clog centrifugal pump, solids submersible pump, diaphragm pump, piston pump or other device configured to move sludge and other viscous, thick or thixotropic materials).

If mechanical means are used to remove the treated compost from the composting unit 10, the suction of the pump or other mechanical device can be connected to a dedicated outlet fitting on the vessel portion 14 (not shown). Alternatively, the suction of the pump (or the pump itself if a submersible or similar pump is used) can be directly placed into the composting unit 10. For example, the suction of the pump can be placed through a cover opening 17, a hatch 18, other accessway or the like. In other embodiments, the treated compost can be transferred out of the composting unit 10 without using a pump. For example, the compost can be removed by simply scooping it out of the hatch 18 or the top of the vessel portion 18 after the cover 14 is removed or through one or more bottom or side openings.

According to some embodiments, water or other liquid that enters the clarifier tank 40 or that is collected in a collection structure 34, a condensation collection member 24 and/or the like can undergo some level of treatment. For example, as discussed, the liquid can pass through a screen or other separation membrane. Alternatively, the liquid can be allowed to naturally separate into two or more different layers. In yet other embodiments, the liquid can undergo chemical and/or biological treatment. After the liquid has been treated, it can be selectively returned to the composting unit 10 (e.g., randomly, according to a timed manner, etc.) to maintain a desired moisture level. Alternatively, the liquid can undergo additional treatment, can be sewered, can be used in other processes and/or the like. Further, solids or other materials removed from the liquid (e.g., using a clarifier tank 40, a subsequent process, etc.) can be returned to the composting unit 10, landfilled and/or the like.

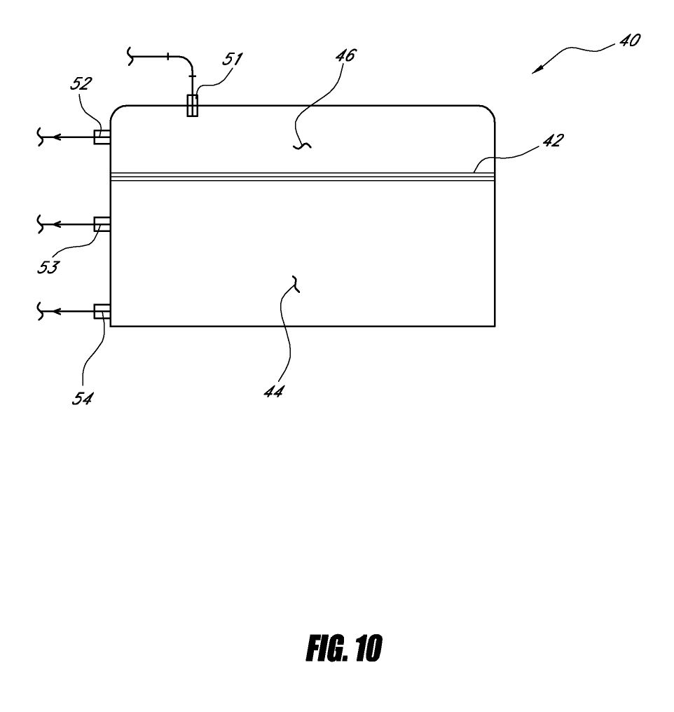

With reference to FIG. 11, another embodiment of a composting unit 400A includes a plurality of openings 420 positioned along the outside of a main vessel portion 414. The openings 420 can be shaped, sized and otherwise configured to permit air or other fluid to enter the interior of the composting unit 400A. This can be especially desirable if an aerobic composting process is being utilized to treat the waste materials fed into the composting unit 400A. In certain embodiments, air or other fluid passing through the main vessel portion 414 can also improve the mixing characteristics within the interior of the composting unit 400A.

In some embodiments, the composting unit 400A includes or is in fluid communication with a suction pump or other vacuum source (not shown) in order to assist in drawing air or other fluid through the plurality of openings 420 located along the vessel portion 414. For example, in FIG. 11, a suction or vacuum source (e.g., pump, compressor, etc.) can be positioned downstream of the outlet 402 (e.g., ventilation passage). As a result, air can be drawn into the interior of the composting unit 400A through one or more of the plurality of openings 420. In other arrangements, a suction or vacuum source can be positioned at one or more other locations (e.g., the interior of the composting unit 400A), either in lieu of or in addition to a suction source at the downstream end of the outlet 402.

In the illustrated embodiment, the openings 420 are located toward the vertical center of the main vessel portion 414. Further, the openings 420 are arranged in a generally zigzag pattern. However, it will be appreciated that the quantity, location, spacing, general orientation and other characteristics of the openings 420 can vary from what is depicted in FIG. 11 and discussed herein. For example, the openings 420 can be dispersed over a larger area of the main vessel portion 414 and/or any other portion of the composting unit 400A (e.g., the cover 416, the bottom portion, etc.). In other embodiments, the main vessel portion 414 includes more or fewer openings 420.

In some embodiments, the openings 420 comprise a generally circular shape having an approximate diameter of ⅛, ¼, ½, ¾ or 1 inches. In other embodiments, however, the size of the openings 420 can be lower than ⅛ inch, greater than 1 inch and/or within the above range (e.g., ⅛ to 1 inches). Alternatively, the openings 420 can include one or more other shapes, such as, for example, rectangular, triangular, other polygonal, elliptical, irregular or the like. Moreover, the diameter or other transverse dimension of the openings 420 can be greater or smaller than indicated above. Moreover, a composting unit 400A can be configured so that the size, shape and/or other characteristics of the openings 420 vary from each other.

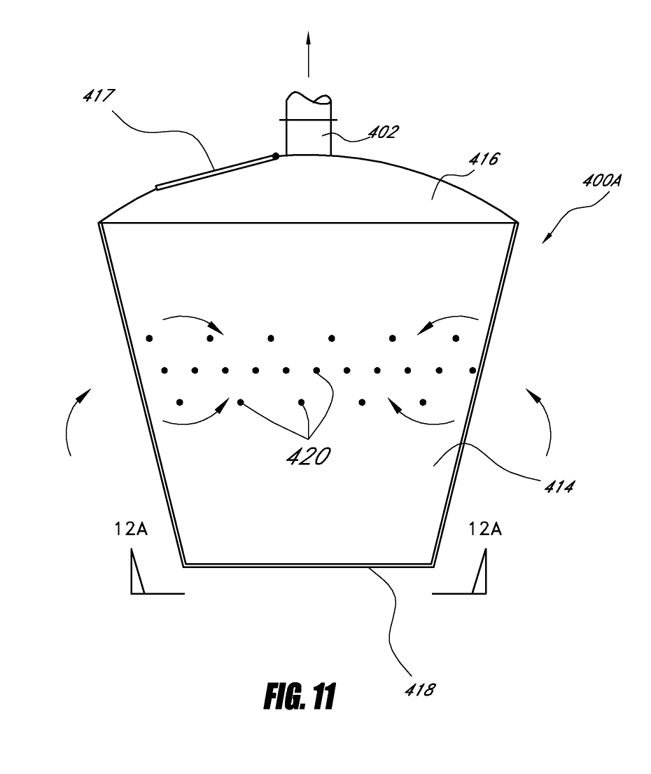

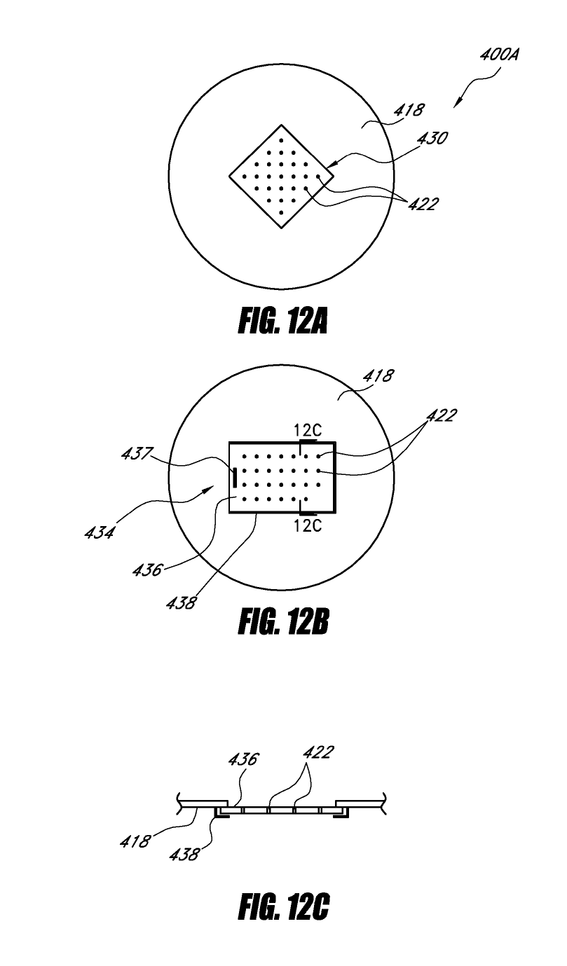

To further enhance the flow of air into the interior of the composting unit 400A, and thus, to improve a desired aerobic environment, openings 422 can be positioned along the bottom 418 of the composting unit 400A. With reference to the bottom view illustrated in FIG. 12A, a plurality of openings 422 can be located within a rectangular region 430, for example, near the center of the composting unit 400A.

The size, shape, location, spacing and other characteristics of the bottom openings 422 can be selected based on the particular use or application (e.g., the type of solids being composted, the water content of the compost, the target operating temperature, the target oxygen concentration within the composting unit 400A, etc.). For example, openings 422 can be located near the periphery of the bottom portion 418, either in lieu of or in addition to the openings 422 disposed near the center as shown in FIG. 12A. In other embodiments, the entire bottom portion 418 comprises openings 422. Alternatively, the composting unit 400A can be configured without any bottom openings 422 at all.

In some embodiments, the bottom openings 422 and/or the side openings 420 (e.g., positioned along the main vessel portion 414) are configured to discharge fluids (e.g., water or other liquid) to the exterior of the composting unit 400A. Thus, such openings 420, 422 can assist the composting process by desirably dewatering the solids being treated. The extent of dewatering through these openings 420, 422 may depend on one or more factors, such as, for example, the size, shape and other details of the openings 420, 422, the type of solids being treated, the water content of the solids being treated, the operating temperature of the composting unit and/or the like.

FIG. 12B illustrates an alternative embodiment of the bottom portion 418 of a composting unit. In the depicted embodiment, the openings 422 are located on a removable plate 436 which is slidably disposed within a receiving system 438. As illustrated in the cross-sectional view of FIG. 12C, the receiving system 438 can comprise outer angles or other members that are configured to receive a perforated plate 436. The removable plate 436 can include a handle 437 or other grasping device to facilitate manipulation of the plate 436 (e.g., removal from the receiving system 438, insertion into the receiving system 438, general handling or maintenance, etc.). The plate 436 and the receiving system 438 can be manufactured from one or more durable materials (e.g., steel, stainless steel, cast iron, other metals, composite materials, etc.) which are configured to withstand the forces, moments, frictional resistance, chemical/biological environments, abrasive contact, wear and tear and/or other conditions to which they may be exposed.

With continued reference to FIGS. 12B and 12C, a removable bottom plate 436 can permit a user to conveniently modify the quantity, size, shape and/or other details of the bottom openings 422. For example, for increased air flow into the interior of the composting unit and/or for increased liquid flow out of the interior of the composting unit, a plate 436 having more and/or larger openings 422 can be selected. Likewise, for decreased air and/or fluid flow, a plate 436 with smaller and/or fewer openings 422 can be used. It will be appreciated that such removable systems can also be used along one or more other portions of the composting unit, such as, for example, the main vessel portion 414 or the cover 416.

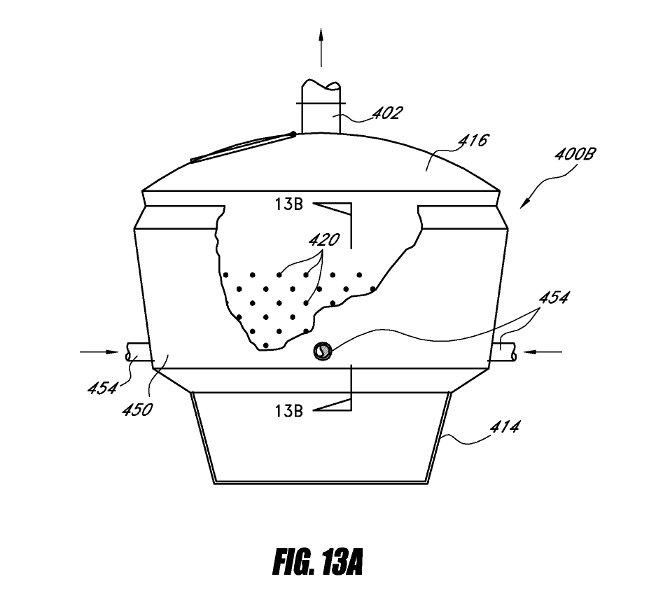

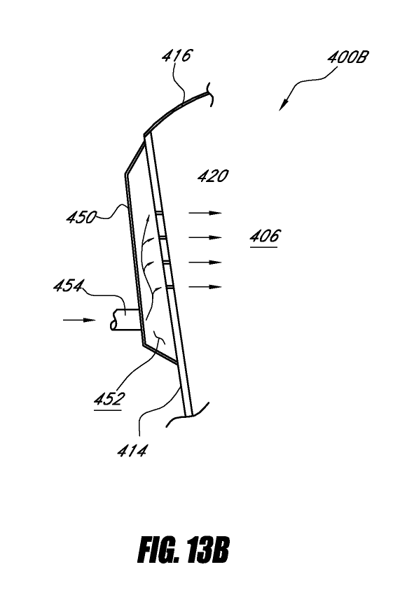

As discussed, to improve aerobic degradation, air, oxygen or other fluids can be delivered into the interior of a composting unit by creating a suction or vacuum at an opening (e.g., the outlet end). In lieu of or in addition to drawing air or other fluid into a composting unit, air or other fluid can be forced across one or more composting unit walls as illustrated in FIG. 13A.

With reference to FIGS. 13A and 13B, the composting unit 400B comprises an outer shell 450 that generally confines a space 452 around an area of the main vessel portion 414. As shown, the shell 450 surrounds a plurality of openings 420, each of which can be in fluid communication with the interior of the composting unit 400B. Further, the shell includes one or more inlets 454 through which air or other fluids can be delivered into the space 452.

In the embodiment depicted in FIGS. 13A and 13B, the shell 450 extends circumferentially around the entire main vessel portion 414. Further, the shell 450 covers only a fraction of the vertical portion of the composting unit 400B. However, it will appreciated that the shape, size, dimension, location, distance from the main vessel portion 414 and other details of the shell 450 can be different than discussed and illustrated herein. For example, in some embodiments, the shell 450 covers substantially the entire outer surface of the main vessel portion 414. Alternatively, the shell 450 can be smaller than shown in FIGS. 13A and 13B (e.g., the shell can extend only partially or intermittently around the periphery of the unit 400B, can encompass a smaller height or vertical distance along the exterior portion of the unit 400B, etc.).

The number of inlets 454 or other connections that a shell 450 comprises can depend on one or more factors, such as, for example, the size of the space 452, the number of openings 420 positioned along the main vessel portion 414, the aerobic demand (e.g., required air flow into the interior of the composting unit 400B), the fluid mechanics associated with such air flow (e.g., head losses) and/or the like. In the illustrated embodiment, the shell 450 includes a total of four inlets 454, which are equally spaced (e.g., 90 degrees) from each other. However, in other arrangements, a shell 450 can include more or fewer inlets 454 as desired or required.

In some embodiments, an air compressor, pump or other positive pressure fluid delivery device can be connected to one or more of the inlets 454 of the outer shell 450. Thus, as illustrated in FIG. 13B, air can be delivered into the space 452 defined by the shell 450 and an adjacent portion of the composting unit wall. Consequently, air delivered into the space 452 can enter the interior 406 of the composting unit 400B through the plurality of openings 420 positioned along the main vessel portion 414. It will be appreciated that pure oxygen or another fluid mixtures can be conveyed into the space 452, either in lieu of or in addition to ambient air.

The flowrate of air or other fluid passing through the inlets 454, and thus, the openings 420, can be modified by modulating the discharge pressure of the compressor, pump or other fluid transfer device (not shown) and/or by using a valve or other flow or pressure regulating device. Moreover, one or more oxygen sensors or the like can be advantageously positioned within the composting unit 400B. This can assist a user to determine if more or less airflow is required. In some embodiments, the air delivery system can be automated so that a substantially constant air flow in being delivered into and/or a desired oxygen concentration in maintained within the interior of the composting unit 400B.

After air or other fluid enters the composting unit 400B, it can be used by microorganisms present therein for the aerobic digestion of the organic materials being treated. Excess air can flow upwards toward the cover 416 of the composting unit 400B where it can be discharged through one or more outlets 402. As discussed in greater detail herein with respect to odor control, fluid discharged through the outlet 402 can be treated using a biofilter, scrubber or other treatment device or method. Alternatively, all or a portion of the fluid discharged through the outlet 402 can be redirected into the space 452 through one or more shell inlets 454.

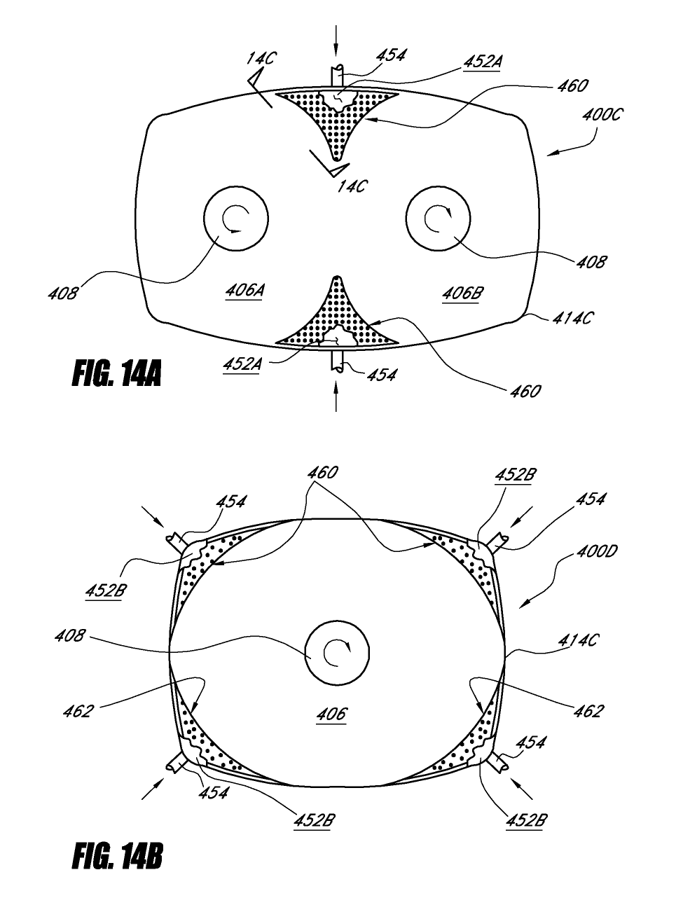

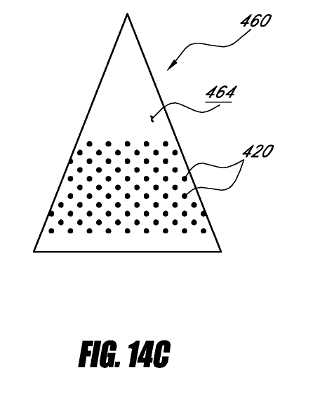

In other embodiments, one or more spaces which receive air or other fluid for distribution into a composting unit are positioned within the interior of the main vessel portion 414. With reference to the top view of FIG. 14A, the composting unit 400C includes two separate zones 406A, 406B, each of which comprises a mixing device 408 (e.g., an auger 408). In order to improve the mixing characteristics of the composting unit (e.g., reduce or eliminate short-circuiting or dead zones), one or more interior baffle units 460 or other members can be used. In FIG. 14A, the depicted composting unit 400C includes two baffle units 460 which are shaped, sized, positioned and otherwise configured to effectively create a substantially circular zone around each auger 408. It will be appreciated, however, that the zones surrounding each auger 408 or other mixing device can comprise a different size, shape and/or other characteristics.

With continued reference to FIG. 14A, the baffle units 460 can also be used as fluid distribution devices to distribute air, oxygen or other fluids into the interior of the composting unit 400C. Similar to the shell 450 discussed and illustrated herein with reference to FIGS. 13A and 13B, the baffle units 460 can comprise an interior space 452. Air or other fluids can be delivered into the interior space 452 through one or more inlets 454. In some embodiments, as shown in FIGS. 14A and 14C, at least a portion of the exterior surface 464 of a baffle unit 460 includes a plurality of openings 420. Thus, air or other fluid delivered into the interior space 452 of a baffle unit 460 can be discharged into the interior zones 406A, 406B of the composting unit 400C. Consequently, such baffle units 460 can be used to aerate the interior of a composting unit 400C while simultaneously improving the mixing characteristics of the system.

FIG. 14B illustrates another embodiment of a composting unit 400D comprising interior distribution baffle units 460. The depicted composting unit 400D includes only a single auger 408 and a single mixing zone 406. However, the distribution baffle units 460 can further improve the mixing characteristics of the vessel by reducing the size of or altogether eliminating dead zones within the interior of the unit 400D. In FIG. 14B, the composting unit 400D comprises a total of four baffle units 460, each of which is positioned along an interior corner of the composting unit 400D. In some embodiments, the baffle units 460 include an interior space 452B which is in fluid communication with an interior 406 of the composting unit 400D through a plurality of openings 420. Thus, air or other fluid discharged into the interior space 452B of a baffle unit 460 through one or more inlets 454 can be distributed into the interior 406 of the composting unit 400D.

Air distribution baffles positioned within the interior of a composting unit 400C, 400D can be used in lieu of or in addition to one or more exterior shells, such as those discussed in relation to FIGS. 13A and 13B.

Further, in order to reduce the undesirable odors associated with the operation of a composting system and/or to comply with one or more environmental or other regulatory requirements, a composting unit can comprise or be integrated with an odor control system. It will be appreciated that any of the composting units disclosed or illustrated herein can be equipped with or be placed in fluid communication with such an odor control system.

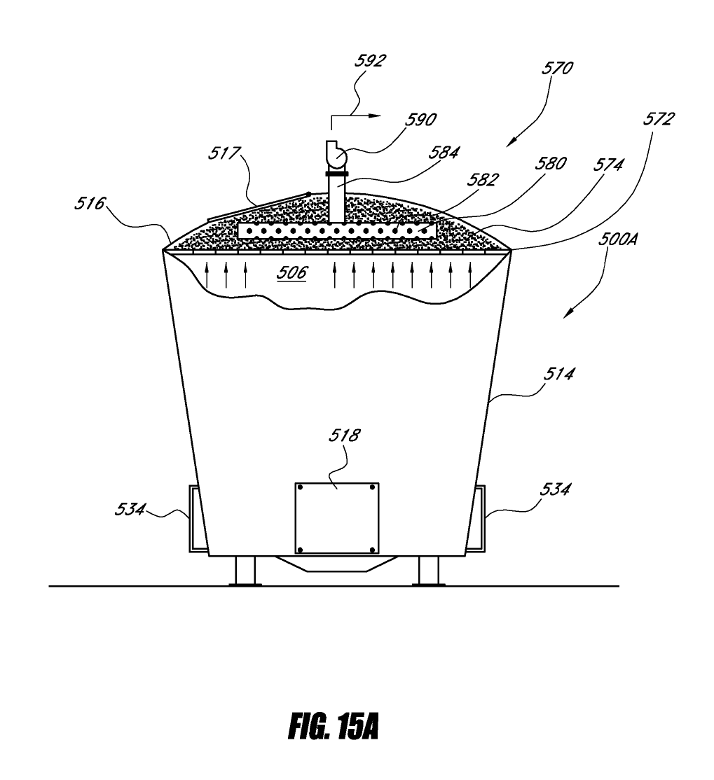

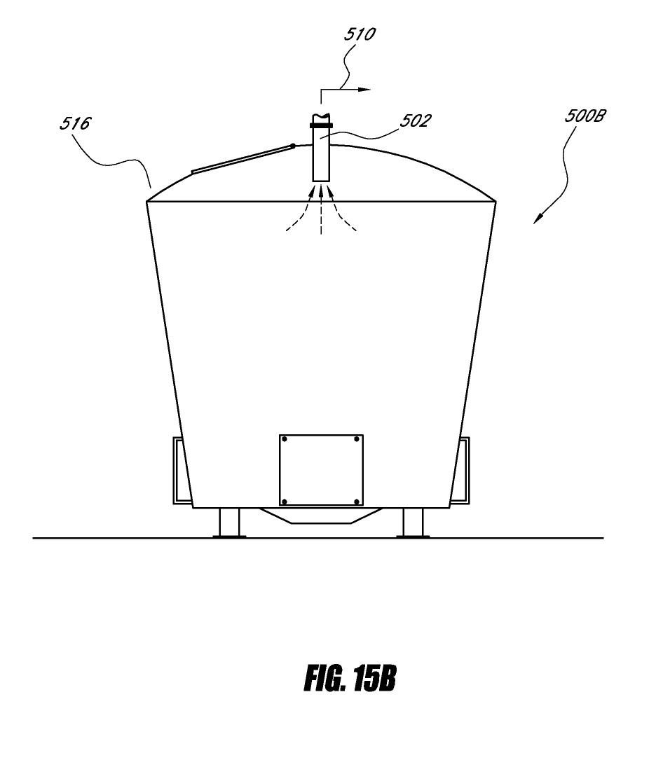

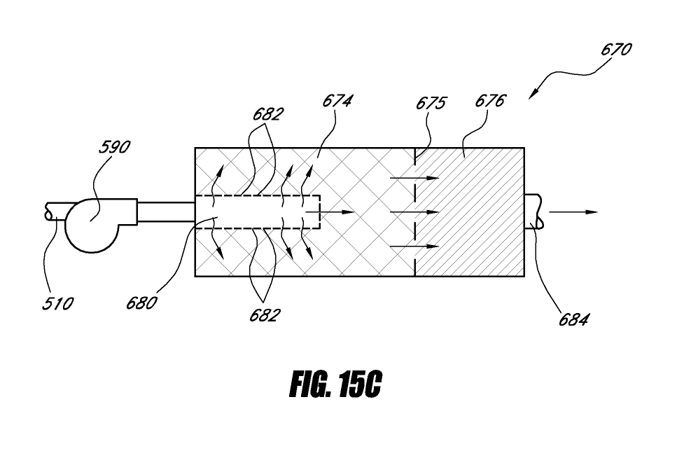

With reference to FIG. 15A, a composting unit 500A can be configured to reduce or eliminate the amount of odorous gases (e.g., hydrogen sulfide, other sulfur-based gases, etc.) and other fluids that leak or otherwise escape from its interior 506. For example, in some embodiments, the composting unit 500A includes gaskets and/or other compressible or resilient members that properly seal hatches 518, 517, accessways or other openings of the unit 500A through which odors and other gases can otherwise escape. Further, other items or features of the composting unit 500A, such as, for example, collection structures 534 positioned along the main vessel portion 514, can be advantageously configured to be air-tight or substantially air-tight.

With continued reference to the embodiment depicted in FIG. 15A, fluid (e.g., foul air) within the interior 506 of the composting unit 500A can generally move towards the cover 516. As shown, the cover 516 can comprise an air treatment system 570 which is configured to advantageously treat a volume of gas (e.g., foul air) before it exits the composting unit 500A into the surrounding environment. In the illustrated embodiment, the air treatment system 570 includes biofilter media (e.g., activated carbon) positioned within the cover portion 516 of the composting unit 500A. Alternatively, other waste air treatment technologies can be used either in lieu of or in addition to biofilter media. Further, it will be appreciated that the biofilter media and/or other air treatment system 570 can be positioned in one or more other locations, either within or outside of the composting unit 500A.

In FIG. 15A, the biofilter media is maintained within the cover portion 516 of the composting unit 500A using a support structure 572. The support structure 572 can comprise a plurality of rigid members, such as, for example, angles, channels, sheets, plates, trusses, beams, columns, platforms and/or the like. The various components of the support structure 572 can be constructed of stainless steel, other metals and metal alloys, fiberglass, aluminum and/or any other suitable material. Preferably, the materials used in the manufacture of the support structure 572 are configured to withstand the weight and other forces to which they may be exposed during the operation of the composting unit 500A. In addition, the support structure components are preferably configured to withstand the normal operating conditions within the composting unit 500A, such as, for example, pH and pH fluctuations, corrosion, abrasion, temperature and temperature fluctuations and/or the like.

As illustrated in FIG. 15A, one or more layers (e.g., packed bed, activated carbon, etc.) of biofilter media 574 are positioned on and supported by the support structure 572. In some embodiments, the biofilter media 574 comprises a high surface area material (e.g., plastic packing, plastic balls and other shapes, plastic peanuts, etc.). The biofilter media 574 is preferably shaped, sized and otherwise configured to promote the growth of microorganisms on its surface. The microorganisms can be generally anaerobic, aerobic and/or anoxic depending on how the composting unit is being operated. Once microorganisms grow and become established on the surface area of the biofilter media 574, air flowing past the media 574 can be partially or completely treated. In some embodiments, organic and/or inorganic pollutants and other substances present within the air can be transferred to the biofilter media 574. The microorganisms can then degrade or convert these pollutants and other substances into inert materials or products which are less offensive or less undesirable.

The treatment system can further include a collection member 580 which, in the illustrated embodiment, comprises a perforated pipe 580 or other conduit. A plurality of openings 582 on the pipe 580 are configured to receive the treated air or other fluid and deliver it to a downstream outlet pipe 584. A pump or other fluid transfer device 590 positioned downstream of the perforated pipe 580 and the outlet 584 provides the necessary suction to remove the treated air from the interior 506 of the composting unit 500A. Treated air or other fluid exiting the composting unit 500A can be discharged to the atmosphere or can undergo additional conveying and/or treatment as needed or required.

As discussed, one or more other types of pollution control technologies can also be used to treat the air or other fluid before it is discharged from the composting unit 500A. For instance, biotrickling filters, bioscrubbers, activated carbon scrubbers and/or the like can be used.

In addition, it will be appreciated that the energy required to move a volume to untreated air through the treatment system 570 can be provided by a pump, compressor or other fluid transfer device placed in fluid communication with (e.g., located upstream of) the treatment system 570.

In some embodiments, an air treatment system 570, such as those described herein or variations thereof, can facilitate the dewatering of the solids being composted. In turn, this can generally improve the overall composting process and/or reduce operating costs. For example, as discussed, the cover portion 516 of the composting unit 500A illustrated in FIG. 15A can be configured to promote the condensation of evaporated water thereon. At least a portion of this volume of water can be collected and removed from the composting unit (see FIG. 7). In addition, a volume of humid air can be removed from the composting unit as part of the air treatment system. The water or other liquid removed as part of the air treatment system can be collected in traps (not shown) or other devices positioned along the downstream air treatment piping system. The collected water or other liquid can then be discharged into a sewer or be conveyed to a treatment device or system for additional processing.

FIG. 15B illustrates another embodiment of a composting unit 500B. In this embodiment, the composting unit 500B does not comprise an internal treatment system 570. Instead, untreated air or other fluid is collected at or near the cover portion 516 of the composting unit 500B. From there, the untreated air can be removed from the interior of the composting unit 500B through an outlet pipe 502 and a downstream piping system 510 (depicted as an arrow for simplicity). In some embodiments, the untreated air is delivered to one or more external treatment systems (e.g., biofilters, biotrickling filters, bioscrubbers, activated carbon tanks, etc.). The external treatment system can be part of a facility's general pollution control system which accepts and treats foul air from one or more other processes.

One embodiment of an external treatment system 670 is schematically illustrated in FIG. 15C. As shown, a fluid transfer device 590 (e.g., pump, blower, compressor, etc.) is configured to deliver a volume of untreated air from the discharge piping 510 of a composting unit (FIG. 15B) to an upstream end of the treatment system 670. In the depicted embodiment, untreated air is first discharged into a perforated pipe 680 or other distribution member.

Untreated air is forced out of a plurality of openings 682 located on the perforated pipe 680. As illustrated in FIG. 15C, the perforated pipe 680 is positioned within a bed of biofilter media 674, activated carbon and/or the like. Thus, air exiting the openings 682 of the perforated pipe 680 enter the biofilm media 674 and undergo treatment.

With continued reference to FIG. 15C, the treatment system 670 can additionally comprise one or more secondary treatment or polishing steps 676. Such a secondary treatment or polishing step 676 can be configured to provide additional removal of inorganic and/or organic pollutants, humidity, odors and/or other substances from the air stream. In some embodiments, such a step 676 comprises a biofilter, biotrickling filter, bioscrubber, activated carbon tank and/or the like. Finally, the treated air can be routed to an outlet 684, where it can be discharged to the environment and/or undergo additional conveying and/or treatment. It will be appreciated that treatment systems 670 can comprise more or fewer steps than discussed and illustrated herein. Further, those of skill in the art will appreciate that one or more other treatment processes can be used, either in lieu of or in addition to the treatment processes disclosed in this application.

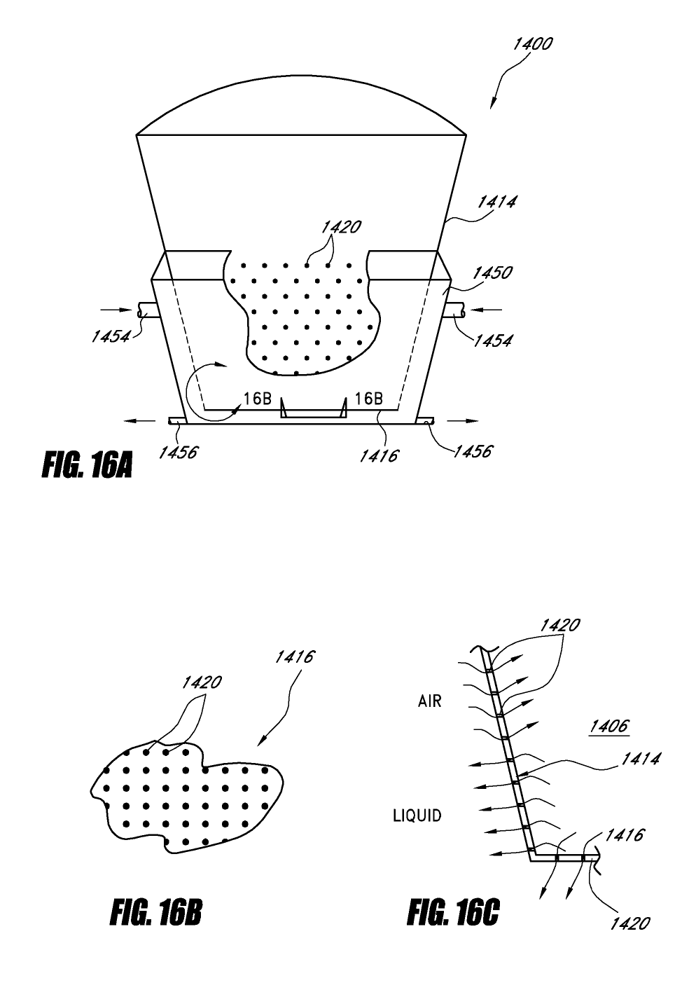

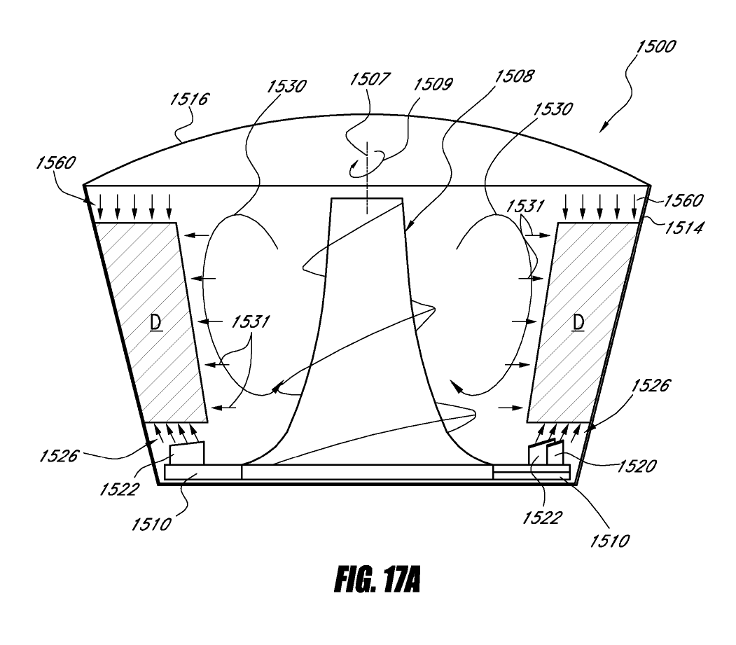

FIG. 16A illustrates one embodiment of a dewatering/composting apparatus 1400 which comprises an outer shell 1450. As shown, the outer shell 1450 and the exterior wall of the main vessel portion 1414 defines a cavity. In some embodiments, the section of the main vessel portion 1414 surrounded by the shell 1450 includes a plurality of openings 1420 which are in fluid communication with the cavity.

With continued reference to FIG. 16A, the outer shell 1450 comprises one or more inlets 1454 which permit air or other fluid to be delivered into the cavity situated between the outer shell 1450 and exterior wall of the main vessel portion 1414. In the illustrated embodiment, the dewatering/composting apparatus 1400 comprises two inlets 1454. However, it will be appreciated that more or fewer inlets 1454 can be connected to the outer shell 1450. In addition, the size, shape, connection location relative to the outer shell 1450 and/or other features or characteristics of the inlets 1454 can vary.

Further, the outer shell 1450 can comprise one or more outlets 1456. According to some embodiments, the outlets 1456 can be located at or near the bottom of the shell 1450 to permit liquids entering into the cavity situated between the outer shell 1450 and the exterior wall of the main vessel portion 1414 to properly drain.

In FIG. 16A, the outer shell 1450 extends around the entire periphery of the dewatering/composting apparatus 1400. In addition, the outer shell 1450 extends only around the lower portion, including the bottom surface, of the main vessel portion 1414. In some embodiments, a dewatering/composting apparatus can comprise two or more different shells 1450. However, in other embodiments the outer shell 1450 can be different than illustrated and described herein. For example, the outer shell can encompass more or less of the exterior surface of the main vessel portion 1414. In some embodiments, the outer shell 1450 surrounds all or a majority of the main vessel portion 1414. Moreover, the shape, size, location and method of connection to the main vessel portion 1414, the material of construction and/or other characteristics of the outer shell 1414 can vary.

According to some embodiments, the quantity, shape, spacing, density, location and/or other details of the openings along the main vessel portion 1414 can be different than illustrated and discussed herein. As shown in FIG. 16B, the bottom surface 1416 of the main vessel portion 1414 can comprise a plurality of openings 1420 either in addition to or in lieu of any openings 1420 located along the side walls of the apparatus 1400.

As illustrated in the cross-section view of FIG. 16C, the openings 1420 located along the sidewall or bottom surfaces of the main vessel portion 1414 can be configured to permit the entry of air or other fluid into the interior 1406 of the dewatering/composting apparatus 1400. Further, the openings 1420 can be advantageously configured to allow liquid (e.g., water, leachate, other fluids, etc.) to exit out of the interior 1406 of the dewatering/composting apparatus 1400. In some embodiments, one or more of the openings 1420 are configured to allow both air to enter into and liquid to exit out of the interior 1406 of the dewatering/composting apparatus 1400.

In some embodiments, a desired back pressure of air or other fluid can be maintained within the interior cavity of the shell 1450 to assist in the delivery of the air other fluid into the interior of the vessel. This is true for any of the embodiments comprising an outer shell described or illustrated herein. This air or other fluid can facilitate in mixing the contents of the dewatering/composting apparatus 1400. In addition, the presence of air or other fluid can help maintain a particular microbial population or other biological environment. For example, oxygen can assist in the aerobic digestion of the materials situated within the dewatering/composting apparatus 1400. Alternatively, gases which contain little or no oxygen can assist in the anaerobic and/or anoxic digestion of materials. One or more pressure regulating valves or other flow or pressure regulating devices can be used to maintain such a fluid backpressure within a desired range.

With continued reference to FIGS. 16A-16C, liquid exiting the interior 1406 of the dewatering/composting apparatus 1400 can be directed to one or more outlets 1456. The outlets 1456 can be advantageously located at or near low points within the shell cavity to allow some, most or all of the liquid to adequately drain. The liquid discharge can be collected into one or more common headers and conveyed for collection and/or additional treatment.

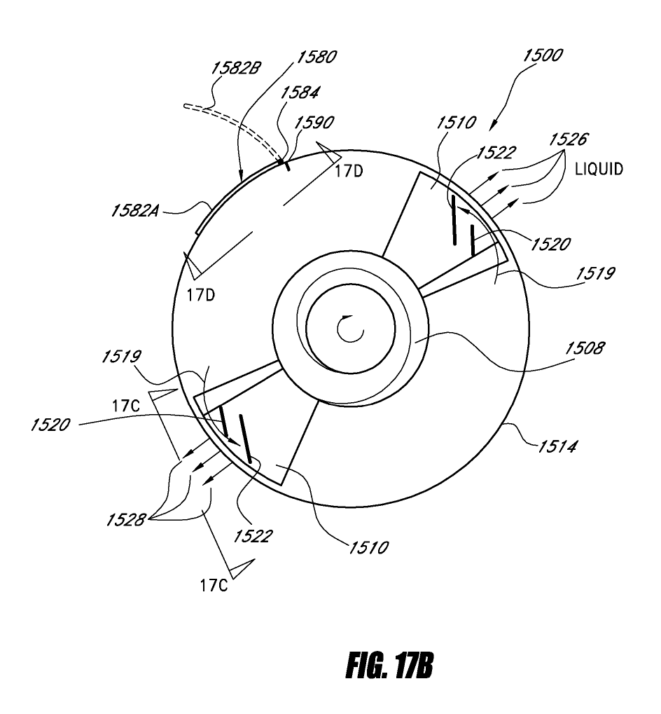

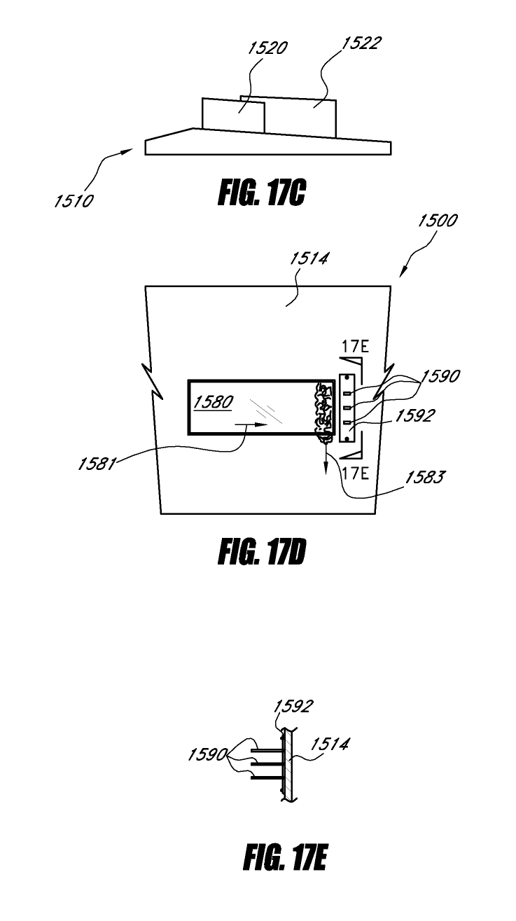

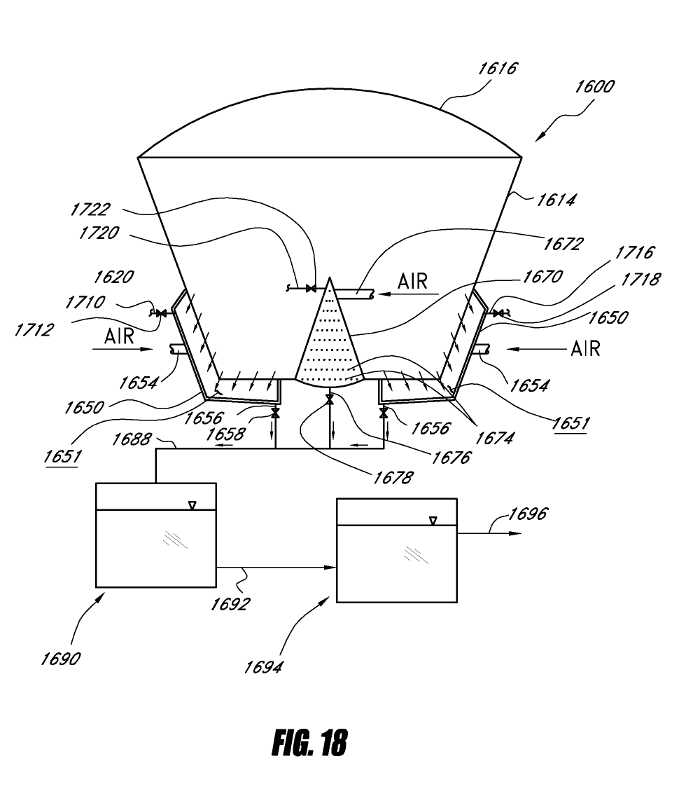

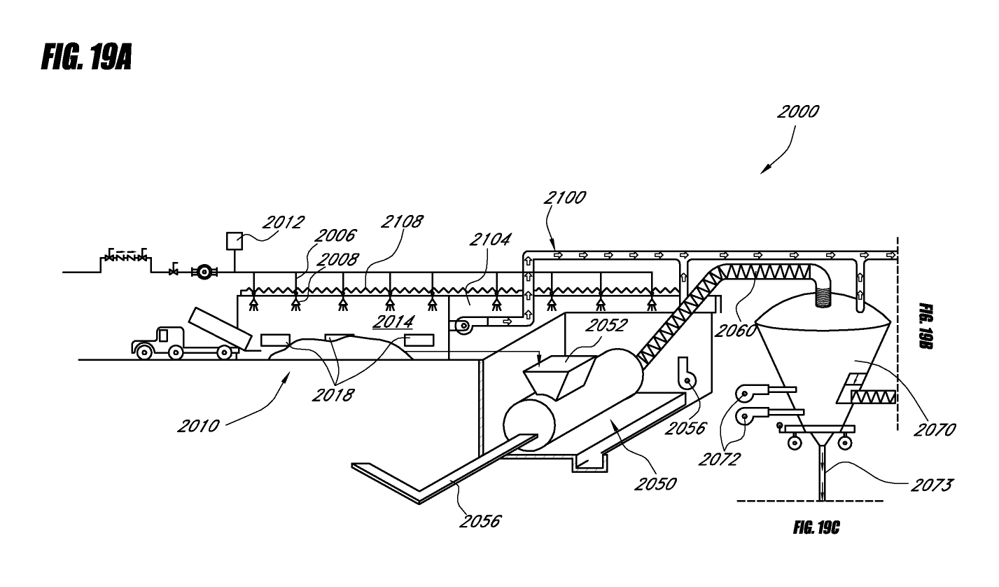

FIG. 17A illustrates another embodiment of a dewatering/composting apparatus 1500. As shown, the dewatering/composting apparatus 1500 can comprise one or more central augers 1508 which are configured to mix the materials contained within the interior of the vessel portion 1514. In some embodiments, as illustrated in FIG. 17A, the auger 1508 includes one or more wing portions 1510 that extend outwardly towards the outer perimeter of the dewatering/composting apparatus 1500. In the illustrated arrangement, the wing portions 1510 are located at or near the bottom portion of the auger 1508. For example, in FIG. 17A, the wing portions 1510 provide the auger 1508 with a generally inverted T-shape. However, in will be appreciated that the wing portions can be located at other locations (e.g., vertical, horizontal, etc.) relative to the auger 1508.

With continued reference to FIG. 17A, the auger 1508 can be configured to turn about a central axis 1507 in a clockwise direction as represented by the arrow 1509. Alternatively, the auger 1508 can be configured to rotate in a counterclockwise direction.

As illustrated in FIGS. 17A-17C, one or more of the wing portions 1510 of the auger 1508 can comprise one or more baffles 1520, 1522. In the depicted embodiment, the baffles 1520, 1522 are secured at or near the outer edges of the wing portions 1510. The baffles 1520, 1522 can be situated on a generally horizontal surface of the wing portion 1510. Alternatively, the baffles 1520, 1522 can be attached to a vertical or any other non-horizontal surface of the wing portion 1510.

In some embodiments, the baffles 1520, 1522 are rigid and/or semi-rigid, and are preferably configured to withstand the forces, moments, chemical environment and other elements to which they may be subjected. Further, the baffles 1520, 1522 can be constructed of one or more corrosion-resistant materials, such as, for example, stainless steel, cast iron, ductile iron, other metals or metal alloys, composites and/or the like. However, in other embodiments, the baffles 1520, 1522 are constructed of one or more non-metallic, semi-rigid and/or any other material. The baffles 1520, 1522 can be attached to the corresponding wing portion 1510 using one or more connection methods or devices, such as, for example, welds, fasteners, pins, snap fittings, slide fittings, adhesives and/or the like. In other embodiments, the baffles 1520, 1522 and the wing portions 1510 of the auger 1508 are manufactured as a unitary member.

As illustrated in FIGS. 17A and 17C, the baffles 1520, 1522 can be oriented in a generally upright position. If two or more baffles 1520, 1520 are positioned on a single wing portion 1510, the baffles 1520, 1522 can be parallel to each other as shown in FIG. 17B. Alternatively, the orientation of the baffles 1520, 1522 relative to the wing portions 1510 and/or to each other can be different than illustrated and discussed herein.

Waste materials (e.g., solid waste, dairy waste, industrial waste, manure, sludge, slurry, etc.) contained within the dewatering/composting apparatus 1500 can be moved as the auger 1508 rotates. With continued reference to FIG. 17B, as the auger 1508 turns, a volume of waste materials located near the bottom of the dewatering/composting apparatus 1500 can be channeled between the baffle 1520 and the interior wall of the vessel portion 1514 of the dewatering/composting apparatus 1500. This compression can help dewater the waste materials. Thus, a volume of water or other liquid which is extracted from the waste materials can be removed from the dewatering/composting apparatus 1500 through a plurality of openings located on the outside and/or bottom of the vessel portion 1514 of the dewatering/composting apparatus 1500. In embodiments having two or more wing portions 1510, such as the one illustrated in FIGS. 17A-17E, the compression can simultaneously occur at two or more interior portions of the dewatering/composting apparatus 1500.

With continued reference to FIG. 17B, the waste materials which pass between the first baffle 1520 and the interior wall of the vessel portion 1514 next encounter a second baffle 1522. Depending on the shape, size, dimensions, hydraulic design/configuration and other features of the dewatering/composting apparatus, such waste materials can be diverted upwardly as illustrated by the arrows 1526 (FIG. 17A). However, in other embodiments, the waste materials can be directed and/or re-directed in one or more other directions. Consequently, a wing portion 1510 of an auger 1508 can comprise more or fewer baffles 1520, 1522 as needed or required. Such baffles can be used to dewater, redirect, compress, mix and/or otherwise affect the waste materials contained within a dewatering/composting apparatus 1500.

In addition, waste materials contained within the upper portions of the dewatering/composting apparatus 1500 generally impart a downward, static pressure force on other waste materials situated at lower elevations. In FIG. 17A, such a static pressure force is represented by the arrows 1560. Those of skill in the art will appreciate that a static pressure force will be exerted at other portions of the dewatering/composting apparatus 1500 as well. It will also be appreciated that one or more other types of forces can be exerted on waste materials contained with the dewatering/composting apparatus 1500.

Therefore, as illustrated in FIG. 17A, a volume of waste materials represented as D is situated between generally opposing upwardly and downwardly oriented forces. In the depicted embodiment, such opposing forces are generally acting towards the peripheral outer portion of the interior of the dewatering/composting apparatus 1500. In other embodiments, however, such opposing forces can act at one or more other portions of the vessel interior.

In FIG. 17A, depending on their magnitude, the opposing forces (generally represented by arrows 1560, 1526) can act to compress a volume of the waste materials D. This can, in turn, facilitate in dewatering such waste materials D. The compression can be further aided by centrifugal forces (generally represented by arrows 1531) created by the rotation of the auger 1508. Such centrifugal forces can impart a lateral force on the compressed waste materials D, urging them against the wall 1514 of the dewatering/composting apparatus 1500. Thus, in some embodiments, the upwardly directed forces created by the baffle system of the wing portion 1510 of the auger 1508, the downwardly directed forces created by the static pressure within the vessel and/or the centrifugal forces created by the rotating auger 1508 can assist in compressing a volume of waste materials within the dewatering/composting apparatus 1500.

In some embodiments, the auger 1508 is configured so that the hydraulic effect of the auger on the compressed volume of waste materials D is reduced or eliminated. Thus, as illustrated in FIG. 17A, the auger 1508 can be configured to mix only a portion of the dewatering/composting apparatus 1500. In some embodiments, this can create an outer annular ring of compressed waste materials D which are generally hydraulically isolated from the rest of the waste materials contained within the vessel 1514 of the dewatering/composting apparatus 1500. However, the dewatering/composting apparatus 1500 can be advantageously configured so that even the volume of compressed waste materials D can move relative to the interior wall of the vessel portion 1514.

With reference to FIG. 17B, the dewatering/composting apparatus 1500 can comprise an opening 1580 or other accessway along one or more sections of the vessel portion 1514. In some embodiments, such an opening 1580 can advantageously comprise a door 1582A or other closure member. In the illustrated embodiment, the door 1582A comprises a hinge 1584 which permits the door 1582A to swing between a closed position (represented by 1582A) and an open position (represented in phantom by 1582B). A dewatering/composting apparatus 1500 can comprise one or more doors 1582A or other closure members to help remove a volume of compressed waste materials D which have been generally compressed against the interior of the vessel portion 1514. Such compressed waste materials D can be removed from within the interior of the dewatering/composting apparatus 1500 either continuously or intermittently (e.g., using a batch system).

FIG. 17D illustrates one embodiment of an opening 1580 along the wall of the dewatering/composting apparatus 1500. As shown, the opening 1580 can comprise a rectangular shape and can be configured to encompass a relatively small portion of vessel wall. However, in other embodiments, the opening 1580 can have a different shape, size, location and/or configuration.

In some embodiments, one or more detachment methods or devices can be used to detach, and subsequently remove, a volume of compressed waste materials D at the opening 1580. As illustrated in FIG. 17D, one or more protruding members 1590 can be affixed to the inside of the dewatering/composting apparatus 1500 to assist in sloughing off or otherwise breaking up a volume of compressed waste materials D. In the depicted embodiment, the protruding members 1590 are rigid members (e.g., steel bars, other metallic members, etc.) which are securely fastened to an interior plate 1592. They can extend from the from the vessel wall towards the interior of the dewatering/composting apparatus 1500. For example, in one embodiment, the protruding members 1590 extend approximately 6 inches into the interior of the dewatering/composting apparatus 1500. The protruding members 1590 can be welded, fastened and/or otherwise permanently or removably secured to the plate 1592. In some embodiments, the protruding members 1590 comprise one or more sharpened surfaces that can assist in sloughing off or breaking up the waste materials. It will be appreciated that in other embodiments, the quantity, type, position, spacing, shape, size and/or other details of the protruding members 1590 can vary.

With continued reference to FIG. 17D, as the compressed waste materials move relative to the vessel wall, in a direction generally represented by arrow 1581, they encounter the opening 1580. Since the protruding members 1590 extend toward the interior of the dewatering/composting apparatus 1500, they provide an obstacle that helps break up a volume of compressed waste materials D. Consequently, in some embodiments, a volume of compressed waste materials D breaks away from the rest of the compressed waste materials D and is removed through the opening 1580. It will be appreciated that the protruding members 1590 can be positioned at any other location of the dewatering/composting apparatus 1500. Further, one or more other methods of causing the dewatered waste materials D to slough off can be used (e.g., other mechanical or hydraulic device, etc.).

FIG. 18 illustrates a dewatering/composting apparatus 1600 comprising one or more shells 1650 along the lower and bottom exterior portions of the vessel 1614. As discussed herein, each shell 1650 can define an interior cavity 1651 which can be placed in fluid communication with a plurality of openings 1620 along the vessel wall. According to some embodiments, such openings 1620 can be configured to permit air or other fluid to enter into the interior of the dewatering/composting apparatus 1600. The openings 1620 can also permit, at times simultaneously, water or other liquids to exit from the interior of the dewatering/composting apparatus 1600. Air or other fluid can be delivered into the cavity 1651 of the shell 1650 through one or more inlets 1654. In addition, liquid (e.g., leachate, water, etc.) removed from the interior of the dewatering/composting apparatus 1600 can be collected at the bottom of the cavity 1651 and conveyed to a collection system.

With continued reference to FIG. 18, the dewatering/composting apparatus 1600 can also comprise one or more internal baffles 1670 or other distribution devices. Such a baffle 1670 or other distribution device can comprise a plurality of openings 1674 that are generally configured to distribute air into the interior of the dewatering/composting apparatus 1600 and/or remove liquids (e.g., leachate, water, etc.) from the dewatering/composting apparatus 1600. As with the liquid flowing into the cavities 1651 of the shells 1650, the liquid removed from the baffles 1670 can be conveyed to one or more collection systems for further conveying and/or treatment.

Leachate or other liquid removed from the dewatering/composting apparatus 1600 conveyed into a collection system can be transferred, via gravity and/or a mechanical transfer device (e.g., pump), to one or more treatment processes. For example, in the embodiment illustrated in FIG. 18, the liquid undergoes a two-stage treatment. The treatment can include, without limitation, liquid/solid separation, biological treatment (e.g., activated sludge treatment), chemical treatment, pH balancing and/or the like. In FIG. 18, the discharge from the first treatment phase 1690 is located near the bottom of the tank. This permits the tank of the first treatment phase 1690 to remain pressurized by the air delivered into the cavities 1651 and/or the baffles 1670.

With continued reference to FIG. 18, as well as other embodiments discussed or illustrated herein that comprise a shell (e.g., FIGS. 13A, 14A, 14B, 16A, etc.), the shells 1650 and/or the baffles 1670 can include a flushing system to remove solids and other materials from the respective cavities 1651 of the shells 1650 and the interior portions of the baffles 1670. For example, a washwater system 1710 can be configured to selectively deliver water, washwater, air and/or other fluid to the inside of the cavity 1651 of the corresponding interior portion of a baffle 1670. The resulting cleaning surge can be removed from the cavities and/or the baffles in the same or similar manner that leachate and/or other liquids exit from the interior of the dewatering/composting apparatus 1600.

The simultaneous delivery of air or other fluid into the shells 1650 and/or baffles 1670 can also maintain the openings into the interior of the dewatering/composting apparatus 1600 relatively free of liquids, solids and/or other substances (e.g., fats, oils, etc.).

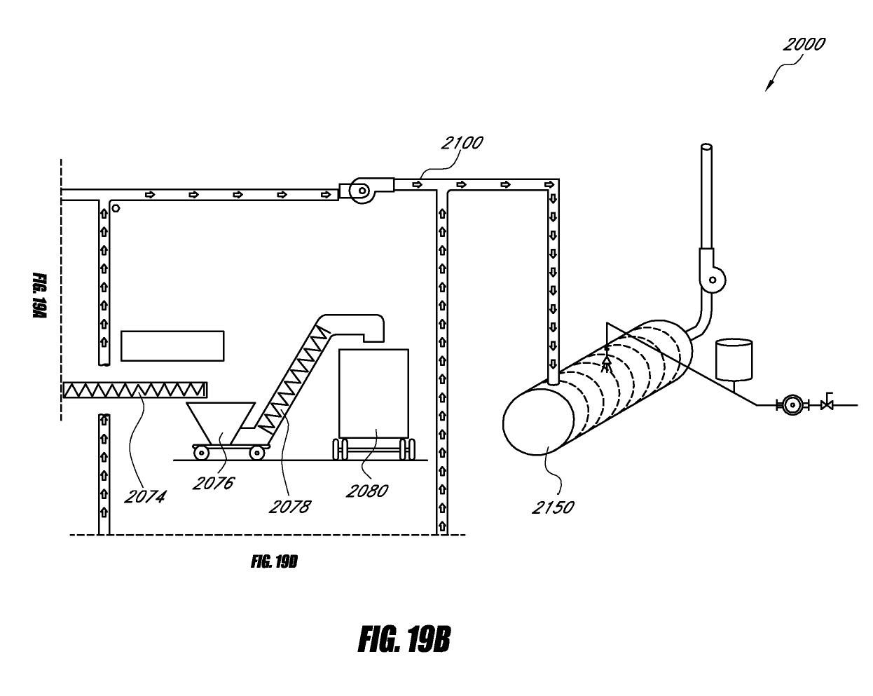

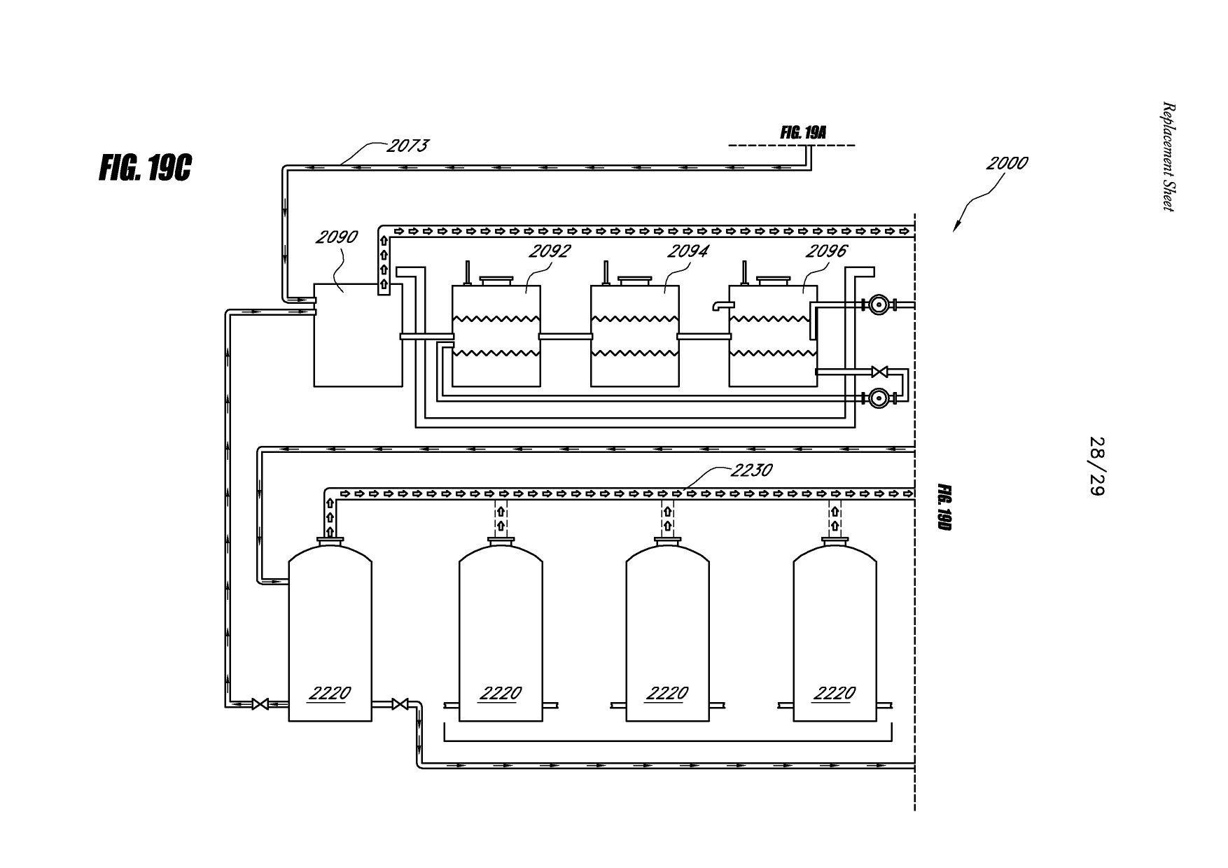

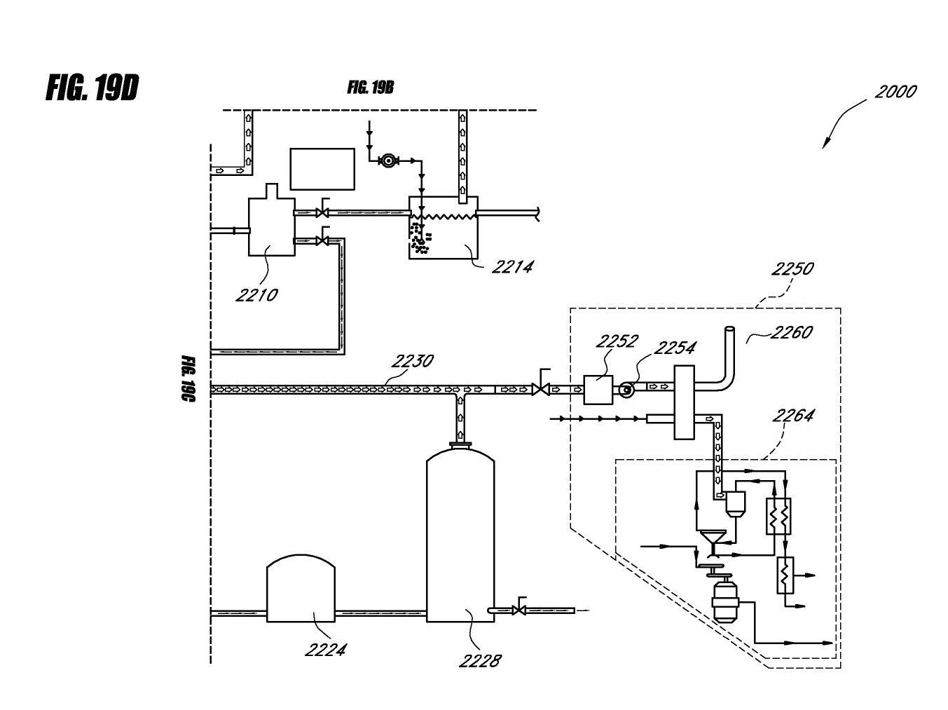

FIGS. 19A through 19D schematically illustrate one embodiment of a solid and liquid waste treatment process 2000 that incorporates a generally facility-wide odor control system. Such a process is particularly well-suited for use with a composing/dewatering unit, trommel screen and/or other treatment steps as discussed and illustrated herein. As such, it should be appreciated that in other arrangements, a treatment system can include more or fewer steps and/or one or more different steps than those disclosed and described herein.

With reference to FIG. 19A, incoming unprocessed waste material can be brought to a solid waste processing or treatment facility 2010. In some embodiments, this involves refuge trucks or other types of disposal vehicles delivering a volume of waste to the facility. In FIG. 19A, the waste is discharged within the interior of a covered building 2014 for odor control purposes. In addition, such a building 2014 can include a spray water misting system 2006 for dust control, odor control and/or any other purpose. As illustrated, one or more chemical additives 2012 can be added to the spray water misting system 2006 upstream of the spray nozzles 2008. The building 2014 can include one or more intakes 2018 (e.g., vents) for collecting air. As shown in FIG. 19A, the collected air can be transferred to a plant-wide air collection system 2100 using one or more exhaust fans 2104 or other fluid transfer devices. Further, a movable odor containment tarp 2108 or other covering can be used to reduce or eliminate the undesirable migration of odors.

With continued reference to the embodiments illustrated in FIG. 19A, the solid waste material can then be transferred to one or more hoppers 2052 or other containers of a trommel screen 2050 or other screening device or apparatus. As discussed, larger materials (e.g., glass bottles, large cardboard items, etc.) can be removed from the trommel screen 2050 via a belt conveyor 2056. Waste materials passing through the trommel screen 2052 can be delivered to the inlet of one or more composting units 2070 via one or more conveyors 2060 or other devices. In the illustrated embodiment, the composting unit 2070 includes two blowers 2072 or other fluid transfer devices that are configured to provide air within the interior of the unit 2070. However, it will be appreciated that a facility 2000 can comprise more or fewer fluid transfer devices 2072.

As illustrated in FIG. 19A, air collected from the trommel 2052 area and the composting unit 2070 can be collected and discharged into the plant-wide air collection system 2100 using exhaust fans 2056 or other fluid transfer devices.

With reference to FIGS. 19A and 19B, composted solids can then be removed from the composting unit 2070 and transferred to a hopper 2076 using one or more conveyor systems 2074. From the hopper 2076, composted solids can be deposited into a compartment of a solid compost transport vehicle 2080 using one or more conveyor systems 2078 or other devices or systems. As discussed, the transport vehicle 2080 can then haul the composted solids off-site (e.g., for landfilling, agricultural spreading, further treatment, packaging, etc.). Preferably, odors coming off the conveyor systems 2074, 2078, the hopper, the transport vehicle station and/or any other portion of the facility 2000 can be collected and directed into the plant-wide air collection system 2100 as depicted in FIGS. 19A and 19B.

As shown in FIGS. 19A and 19C, in some embodiments, liquid waste (e.g., leachate) collected in the composting unit 2070 can be transferred to a pre-treatment tank 2090 via a liquid waste piping system 2073. Liquid waste collected in the pretreatment tank 2090 can be subsequently routed to a number of tanks for specific types of treatment and/or processing. For example, in the illustrated arrangement, liquid waste exiting the pretreatment tank 2090 is routed to a solid separator unit 2092, a grease separator unit 2094 and a final water storage tank 2096. It will be appreciated that other treatment and/or processing steps can be included, either in lieu of or in addition to the steps depicted in FIG. 19B. As with previous steps or processes, air collected from the pretreatment tank 2090 is collected and routed to the plant-wide air collection system 2100.

With reference to FIGS. 19C and 19D, water discharged from the water storage tank 2096 can be pumped to a water discharge test port 2210. From there, a portion of the water or other liquid can be routed to an aeration tank 2214, where it is aerated with ambient air and discharged to a sewer pipe. Air from the aeration tank 2214 can be collected and delivered to the plant wide air collection system 2100. The remainder of the water or other liquid from the water discharge test port 2210 can be routed to one or more acidic processing tanks 2220. As illustrated in FIGS. 19C and 19D, from the acidic processing tanks 2220, the stored water or other liquid can be delivered to a pretreatment tank 2090 and/or a buffer tank 2224 and a subsequent bio-gas reactor tank 2228. In the illustrated embodiment, water or other liquid exiting the bio-gas reactor tank 2228 can be discharged to a public sewer. However, it will be appreciated that water or other liquids can be subjected to a different treatment and/or handling scheme than illustrated and discussed herein.

With continued reference to the embodiment illustrated in FIGS. 19C and 19D, air collected from the acidic processing tanks 2220 and/or the bio-gas reactor tank 2228 can be discharged into a single collection system 2230 and delivered to gas processing station 2250. In some embodiments, the bio-gas and other fluids entering the gas processing station 2250 are first treated in a hydrogen sulfide station 2252 to substantially remove hydrogen sulfide and/or other sulfur and non-sulfur compounds. From the hydrogen sulfide station 2252, a gas booster pump 2254 or another fluid transfer device can convey the gas to a mixing manifold 2258, where the gas is either flared off (e.g., at a methane gas emergency flare station 2260) and/or transported to a combustion/power generation station 2264. The combustion/power generation station 2264 can be configured to use the bio-gas to generate power for the facility and/or to feed a local power grid.

With continued reference to FIG. 19B, waste air collected from the various treatment and/or handling processes is collected into main header 2100 and delivered to a biofilter treatment system 2150. It will be appreciated that the air can be delivered to one or more other treatment steps or apparatuses, either in lieu of or in addition to the biofilter treatment system 2150. The biofilter treatment system 2150 can be configured similarly to the treatment systems discussed and illustrated herein. However, in other embodiments, the biofilter treatment system 1150 can be differently configured than disclosed herein. As shown in FIG. 19B, treated air can be discharged (e.g., vented) to the atmosphere.

The above embodiments of a composting and/or dewatering/composting apparatuses can be used to dewater and/or treat any kind of waste or non-waste materials, such as, for example, solid waste, dairy water, animal waste, industrial waste, sludge, slurry, manure and/or the like. In addition, such apparatuses can be used to simply dewater such materials, either alone or in conjunction with digestion and/or composting.

The dewatering of such materials can help reduce the need for large acreage which is sometimes required to remove a desired level of moisture from such waste materials. In addition, substantial amounts of dewatering time can be reduced when compared to traditional dewatering processes. Thus, the embodiments of the dewatering/composting apparatuses described herein can be simply used as a preliminary conditioning step before composting and/or other treatment steps are performed to a volume of waste materials.

In some embodiments, the dewatered waste materials (e.g., dairy waste, manure, sludge, etc.) can then be composted using one or more other methods (e.g., static windrows, land pile composting, etc.). Alternatively, the dewatered solids can then be transferred to one or more other dewatering/composting apparatuses for additional dewatering and/or composting. In yet other embodiments, waste materials can be dewatered and composted in a single dewatering/composting apparatus.

As discussed herein, in some embodiments, the temperature rise resulting from the aerobic degradation (e.g., composting processes) occurring within such dewatering/composting apparatuses, can help enhance the dewatering of such waste materials.

Although this invention has been disclosed in the context of certain preferred embodiments and examples, it will be understood by those skilled in the art that the present invention extends beyond the specifically disclosed embodiments to other alternative embodiments and/or uses of the invention and obvious modifications and equivalents thereof. In addition, while a number of variations of the invention have been shown and described in detail, other modifications, which are within the scope of this invention, will be readily apparent to those of skill in the art based upon this disclosure. It is also contemplated that various combinations or subcombinations of the specific features and aspects of the embodiments may be made and still fall within the scope of the invention. Accordingly, it should be understood that various features and aspects of the disclosed embodiments can be combined with or substituted for one another in order to form varying modes of the disclosed invention. Thus, it is intended that the scope of the present invention herein disclosed should not be limited by the particular disclosed embodiments described above, but should be determined only by a fair reading of the claims that follow.

What is claimed is:

1. An apparatus for dewatering and composting a volume of waste, the apparatus comprising:
a vessel portion comprising an upper end, an exterior surface, an interior surface and a plurality of openings, the openings extending from the interior surface to the exterior surface;
a cover portion attached to the upper end of the vessel portion, the cover portion and vessel portion defining an interior cavity;
at least one sleeve portion positioned at least partially along the exterior surface of the vessel portion, the sleeve portion comprising an inlet, the sleeve portion and the exterior surface defining a space; and
at least one mixing member positioned within the interior cavity;
wherein at least some of the openings in the vessel portion are in fluid communication with the space; and
wherein the openings are configured to permit fluids to discharge therethrough, both from the interior cavity to the space and from the space to the interior cavity.

2. The apparatus of claim 1, wherein the mixing member comprises an auger.

3. The apparatus of claim 2, wherein the auger is generally cone-shaped.

4. The apparatus of claim 1, wherein the inlet of the sleeve portion is configured to be placed in fluid communication with an air supply.

5. The apparatus of claim 1, wherein the sleeve portion further comprises at least one outlet, the outlet being configured to collect liquid discharged from the openings.

6. The apparatus of claim 1, wherein the cover comprises a ventilation passage, the passage configured to convey a volume of gas out of the interior cavity.

7. A dewatering system for use in a composting apparatus, the dewatering system comprising:
a vessel portion having side walls and a bottom surface, at least one of the side walls or the bottom surface of the vessel portion comprising a plurality of openings configured to permit a liquid to discharge therethrough; and
at least one liquid collection member configured to collect liquid discharged from the openings;
wherein the operation of a mixing member within an interior of the vessel portion facilitates the flow of liquid through the openings.

8. The system of claim 7, wherein the liquid collection member is generally positioned along an exterior area of the vessel portion.

9. The apparatus of claim 7, wherein the vessel portion comprises generally cylindrically-shaped side walls.

10. The apparatus of claim 7, wherein openings on the bottom surface of the vessel portion are located on a removable member, the removable member being configured to be selectively secured to and removed from the vessel portion.

11. A method of dewatering a volume of waste materials placed situated within a composting apparatus, the method comprising:
providing a composting apparatus, the composting apparatus comprising a vessel portion and a cover member situated generally above the vessel portion, the vessel portion and the cover member defining an interior cavity, wherein the vessel portion comprises an interior surface and a plurality of openings being in fluid communication with the interior cavity;
operating at least one mixing member, the mixing member having an axis and being positioned at least partially within the interior cavity, the mixing member comprising a lower base portion, the lower base portion comprising at least one baffle; and
accessing a volume of waste materials by opening a closure member situated along the vessel portion;
wherein operating the mixing member causes a volume of waste materials to be compressed against a portion of the interior surface of the vessel portion.

12. The method of claim 11, wherein the mixing member comprises an auger, and operating the mixing member comprises causing the mixing member to rotate about the axis.

13. The method of claim 11 further comprising providing at least one protruding member along the vessel portion to facilitate collection of a volume of waste materials.
